(19) United States
(12) Patent Application Publication
(10) Pub. No.: US 2004/0265948 A1
(43) Pub. Date: Dec. 30, 2004

(54) Microbial method for hydrolysis and oxidation of androst-5-ene and pregn-5-ene steroid esters

(76) Inventors: Michael Jon White, Portage, MI (US); Doris M. Beck, Kalamazoo, MI (US); Peter Guillaume Marie Wuts, Mattawan, MI (US); Ivan Gale Gilbert, Kalamazoo, MI (US)

Correspondence Address: PHARMACIA & UPJOHN, 301 HENRIETTA ST, 0228-32-LAW, KALAMAZOO, MI, 49007, US

(21) Appl. No.: 10/842,209

(22) Filed: May 10, 2004

Related U.S. Application Data
(60) Provisional application No. 60/482,916, filed on Jun. 27, 2003.
(60) Provisional application No. 60/483,788, filed on Jun. 30, 2003.

(51) Int. Cl. C12P 33/20; A61K 31/58

(52) U.S. Cl. 435/53

(57) ABSTRACT

A microbial method for hydrolysis and oxidation of androst-5-ene and pregn-5-ene steroid esters is disclosed.

### CROSS REFERENCE TO RELATED APPLICATIONS

This application claims the benefit of U.S. provisional application Ser. No. 60/482,196 filed on 27 Jun. 2003, and 60/483,788 filed on 30 Jun. 2003, under 35 USC 119(e)(i), which are incorporated herein by reference in their entirety.

### FIELD OF THE INVENTION

This invention describes a microbial transformation of 3,7-dihydroxy or 3-hydroxy-7-carboxy substituted 5-ene steroid compounds in which there is concomitant hydrolysis of alkanoyl esters, oxidation of a 3-hydroxy to a 3-ketone and migration of the 5,6 double bond to the 4,5 position. The resultant products are intermediates useful in the preparation of eplerenone and other 7-substituted steroids.

### BACKGROUND OF THE INVENTION

Certain 7-carboxy substituted steroids, for example eplerenone, are well known for their aldosterone antagonist activity and are thus useful in the treatment and prevention of diseases of the circulatory system. U.S. Pat. Nos. 4,559,332 and 5,981,744 and International Publication WO98/25948 describe methods for the preparation of eplerenone and related compounds. However, the advent of new and expanded clinical uses for eplerenone creates a need for improved processes for the manufacture of this and other related steroids.

Microbial transformations of steroid compounds in which there is concomitant hydrolysis of C₁-C₄ alkanoyl esters followed by oxidation of a 3 hydroxy group to the corresponding ketone have been reported (see for example: U.S. Pat. Nos. 4,012,510; 3,379,745; 3,352,923; 3,293,285). However, these biotransformations have heretofore not been applied to 7-substituted steroids. This transformation, if done chemically, requires a number of steps and can lead to epimerization of substituents at C-7.

### SUMMARY OF THE INVENTION

This invention relates to processes for the microbial transformation of 7-substituted steroid compounds of Formula I,

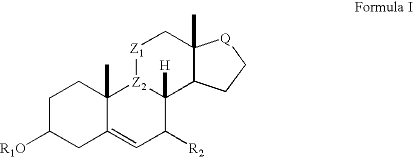

wherein:

R₁ is H or C₁-C₆-alkylC(O)—;

R₂ is —OR, or —C(O)—OC₁-C₆ alkyl

Z₁ is

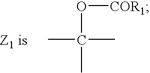

Z₂ is —CH—;

or Z₁ and Z₂ may be taken together to form a carbon-carbon double bond;

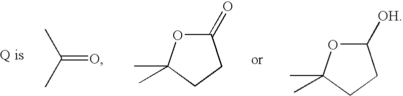

to steroid intermediates of Formula II,

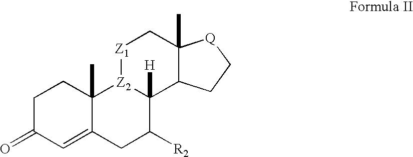

wherein R₂, Z₁, Z₂, and Q are as for Formula I;

when compounds of Formula II are used as intermediates for eplerenone synthesis, R₂ is β-OR₁ or α-C(O)—OC₁-C₆ alkyl.

The compounds of Formula II are useful for the preparation of 7 substitued steroids, especially eplerenone, as described in detail in the Description of Embodiments.

### DESCRIPTION OF THE EMBODIMENTS

### Definitions

In the detailed description, the following definitions are used.

The term “alkyl,” by itself or as part of another substituent, means, unless otherwise stated, a straight or branched chain, or cyclic hydrocarbon radical, or combination thereof. Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)ethyl, cyclopropylmethyl, homologs and isomers of, for example, n-pentyl, n-hexyl, n-heptyl, n-octyl, and the like.

The term “biotransformation” means transformation of chemical compounds within a living system.

The term “Lewis acid” means an electron pair acceptor as defined in McQuarrie, D. A., et. al., General Chemistry, third edition, W.H.Freeman and Company pub., 1991, p. 665.

### DETAILED DESCRIPTION OF THE INVENTION

The inventors have discovered that steroid compounds of Formula (I)

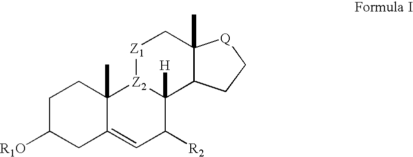

wherein:

R₁ is H or C₁-C₆-alkylC(O)—;

R₂ is —OR₁ or —C(O)—OC₁-C₆ alkyl

Z₁ is

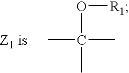

Z₂ is —CH—;

or Z₁ and Z₂ may be taken together to form a carbon-carbon double bond;

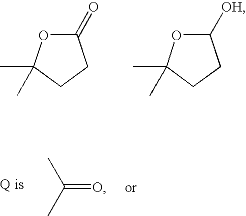

are, unexpectedly, deacylated and subsequently oxidized to form steroid compounds of Formula (II)

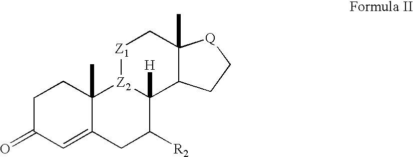

wherein R₂, Z₁, Z₂, and Q are as for Formula I.

The compounds of Formula II are useful for the preparation of 7 substitued steroids, especially eplerenone. When compounds of Formula II are used as intermediates for eplerenone synthesis, R₂ is β-OR₁ or α-C(O)—OC₁-C₆ alkyl.

The biotransformation, surprisingly and unexpectedly, accomplishes in a single operation hydrolysis of acyl groups, selective oxidation of only the 3-hydroxy group and migration of the 5,6-double bond to the 4,5-position. Further, the transformation does not impact the stereochemistry of the substituent at C-7. The biotransformation proceeds through the intermediates I, where R₁ is H, which can also be isolated.

The biotransformation can be achieved with any bacterium belonging to the genus Flavobacterium capable of performing the biotransformation, and in particular Flavobacterium dehydrogenans the strain ATCC 13930. A method for identifying strains capable of performing the biotransformation is illustrated in Example 1. The bacterium may be utilized in the form of an actively growing culture, either in the absence or presence of a water-immiscible organic solvent. Usually, the bacterium is grown in submerged culture under aerobic conditions, using any art-recognized procedure, and steroid transformations performed in situ.

The desired bacterium may be cultured using conditions identified in examples 1-3 using the ingredients specified. Carbon sources may include sugars such as monosaccharides, disaccharides, trisaccharides, hydrolyzed polysaccharides, sugar acids, and sugar alcohols. Preferably a monosaccharide, disaccharide or sugar alcohol is used as the carbon source. More preferably, the monosaccharide glucose (dextrose) is used. The concentration of carbon source may be from about 0.5 g/L to about 40 g/L, but typically from about 2 g/L to about 10 g/L. Nitrogen sources may include nitrogen-containing organic substances such as casein, cornsteep liquor, meat extract, peptone, soy protein hydrolysate, soy flour, and yeast extract, and/or nitrogen-containing inorganic compounds such as nitrates and inorganic ammonium salts. Preferably, the nitrogen-containing organic substance yeast extract and the nitrogen-containing inorganic compound ammonium sulfate are used. Yeast extract may be used at a concentration from about zero g/L to about 25 g/L, but typically about 10 g/L to about 20 g/L. Ammonium sulfate may be used at a concentration from about zero g/L to about 10 g/L, but typically from about 0.5 g/L to about 5 g/L. Other suitable carbon and nitrogen sources are known to those skilled in the art.

Generally a primary and secondary vegetative seed procedure is used in preparation for the bacterial steroid transformation. Alternatively, a primary vegetative seed can be used directly to inoculate bioconversion media. Primary vegetative seed cultures may be incubated for a period of about 24 to about 96 hours (preferably about 48 hours) at a temperature between about 220 and about 37° (preferably about 28°), and a pH between about 5.0 and about 8.0 (preferably between about 6.0 and about 7.5). Secondary vegetative seed medium is inoculated with about 0.1% to about 1.0% (v/v) primary vegetative seed culture, but typically about 0.5% (v/v), and incubated for a period of about 24 to about 96 hours (preferably about 48 to about 72 hours) at a temperature between about 22° and about 37° (preferably about 28°). The pH of the secondary seed medium can be between about 5.0 and about 8.0 (preferably between about 6.0 and about 7.5. The bioconversion medium is inoculated with about 1% to about 10% (v/v) secondary vegetative seed culture, but typically about 5% (v/v), and incubated at a temperature between about 22° and about 37° (preferably about 28°). The pH of the bioconversion medium can be between about 5.0 and about 8.0 (preferably between about 6.0 and about 7.5). Steroid substrates of Formula (I) may be added to the bioconversion medium, dissolved in a minimal volume of water-miscible solvent such as acetone, methanol, ethanol, DMSO or DMF, prior to sterilization and inoculation. It is preferred to use substrates of Formula (I) at a concentration greater than 0.5 g/L, more preferably greater than 1.0 g/L, even more preferably greater than 4 g/L. Alternatively, micronized steroid substrates of Formula (I) may be added to the growing culture between zero hours and about 72 hours post-inoculation (preferably between about 24 hours and about 48 hours). One may also choose to add steroid substrates of Formula (I), dissolved in a water-immiscible organic solvent, to a culture that has been induced for deacylase and 3β-alcohol dehydrogenase activities. Water-immiscible organic solvents such as toluene, branched octane, dichloromethane, octanol, and mixtures thereof may be used at a ratio of about 0.1-2:1 (v/v), solvent:whole beer, but typically about 0.5:1 (v/v). Any 3-ol-Δ⁵-steroid possessing acetate esters can be used to induce these enzyme activities. The concentration of inducer used is from about 1 mg/L to about 100 mg/L, but typically about 10 mg/L. Inducer may be added to the bioconversion medium, dissolved in a minimal volume of water-miscible solvent such as acetone, methanol, ethanol, DMSO or DMF, prior to sterilization and inoculation, or as a micronized slurry between zero hours and about 36 hours post-inoculation, but typically between about 12 hours and 24 hours. Bioconversion of steroid substrates of Formula (1) is allowed to proceed for between about 1 and 5 days, but typically about 2 to about 3 days.

Once the bioconversion of steroid substrates of Formula (I) is complete, steroid products of Formula (II) can be isolated using any one of a number of art-recognized procedures or, more specifically, using the solvents and conditions described in the examples. Preferably, the whole beer is extracted using an organic solvent, such as ethyl acetate, toluene, butyl acetate, or methylene chloride and the deacylated products of Formula (II) are isolated by crystallization. Silica gel chromatography (approximately 50 g of silica per gram of product) may be used to separate the deacylated products of Formula (II) prior to crystallization. The column chromatography and crystallization solvents include solvents such as water, methanol, acetone, butyl acetate, methylene chloride, or combinations thereof. The preferred extraction solvent is methylene chloride; the preferred chromatography solvent is 95% methylene chloride/5% methanol; and, the preferred crystallization solvent is n-butyl acetate.

The products of the biotransformations are useful in the synthesis of 7-substituted steroids and, in particular, eplerenone. Schemes I-VI illustrate the processes of this invention where the products of the biotransformation are compounds of Formula II. In the schemes, illustrative of the invention are steps I-F, II-C, III-B, IV-C, VA and VIA.

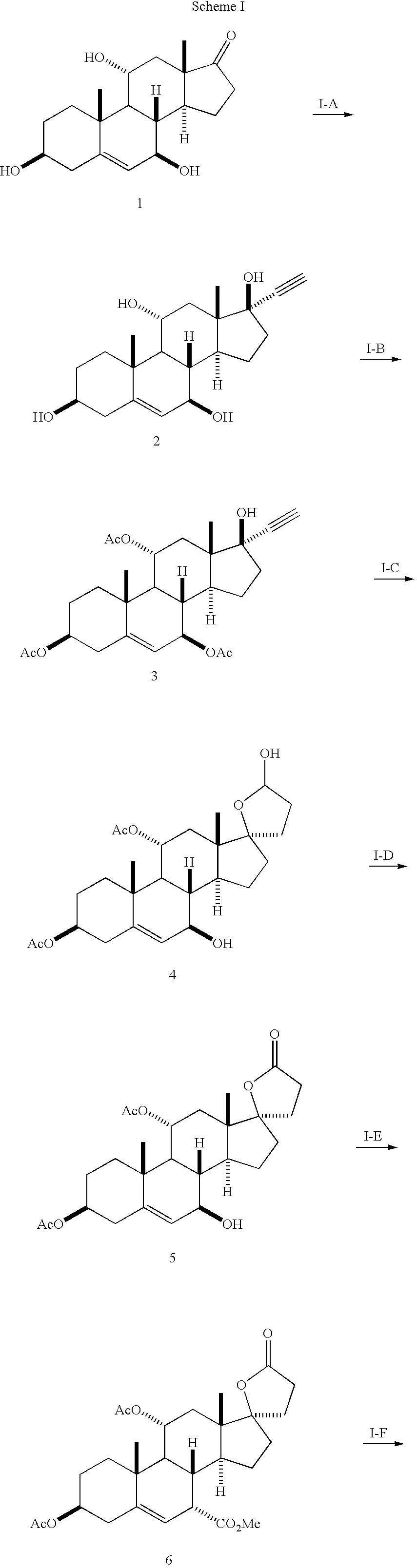

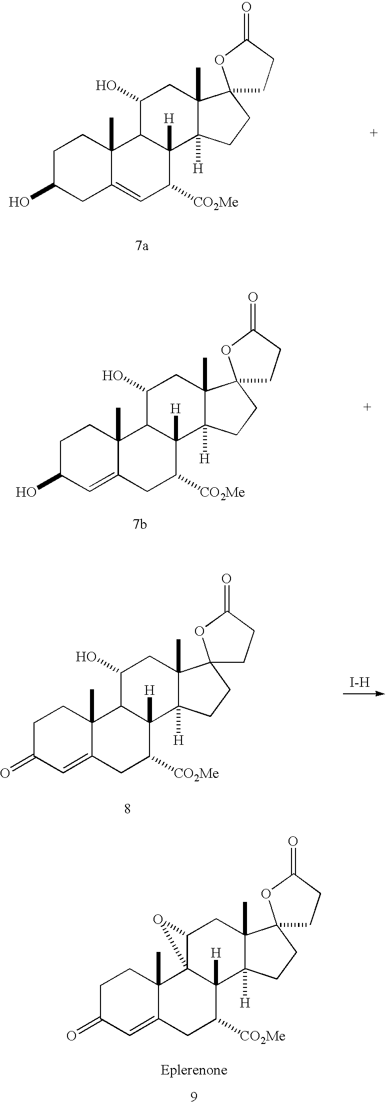

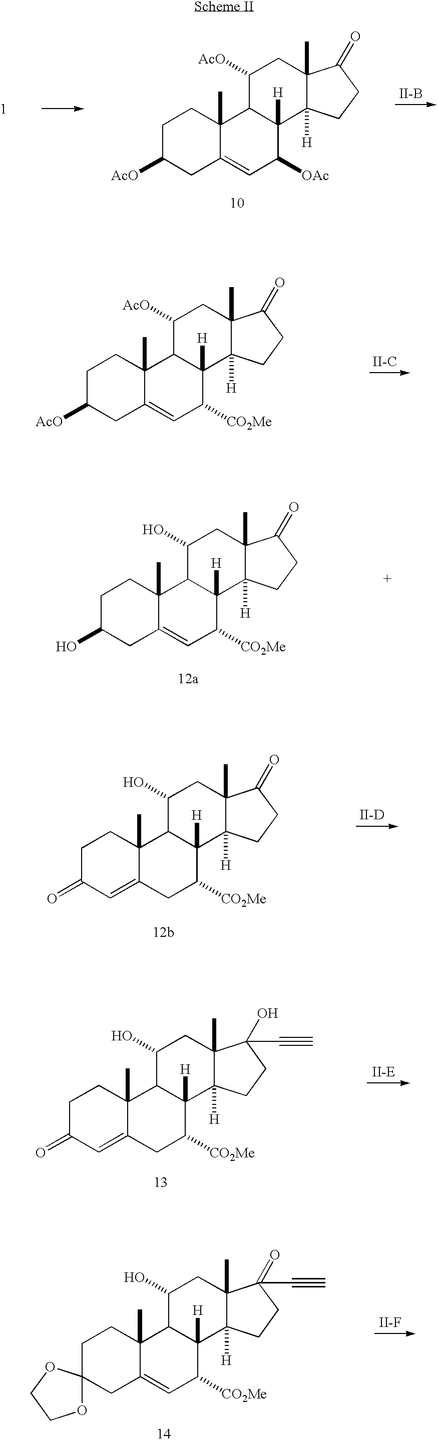

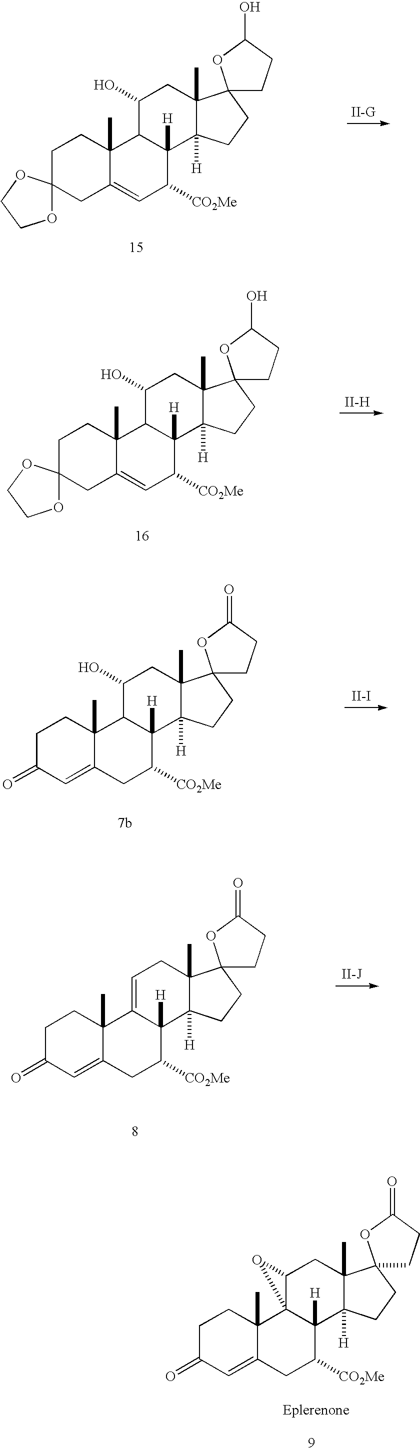

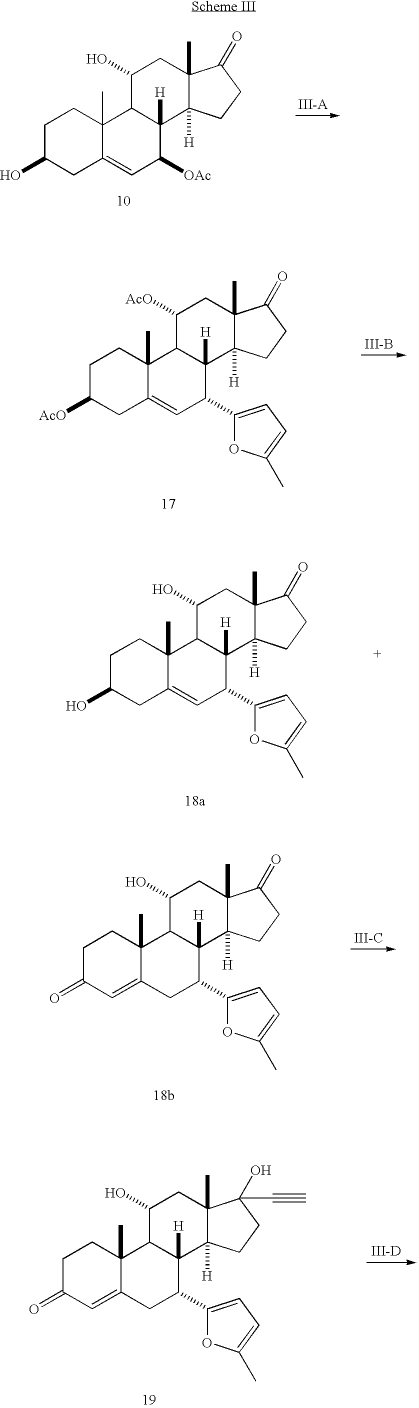

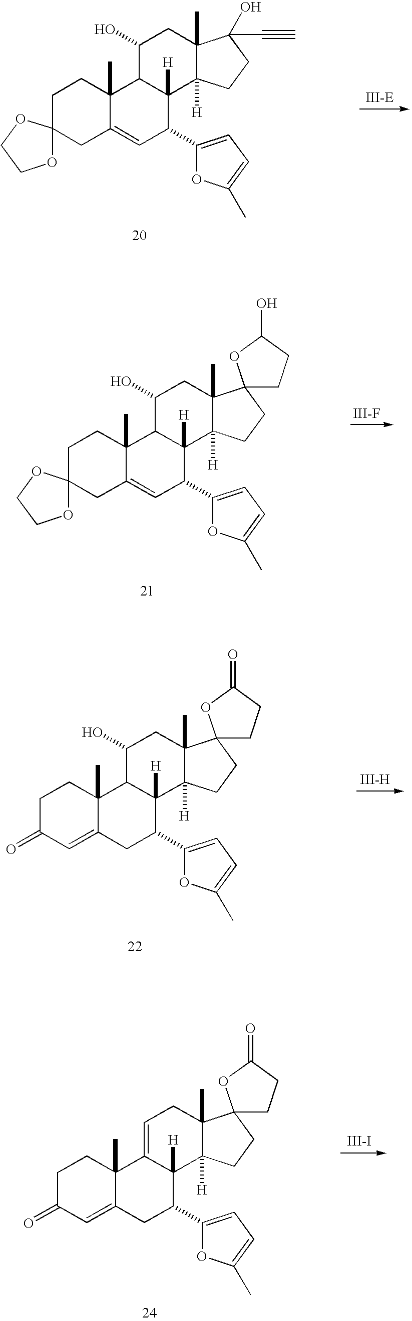

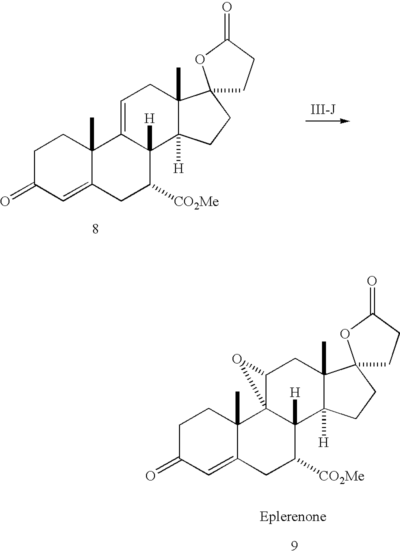

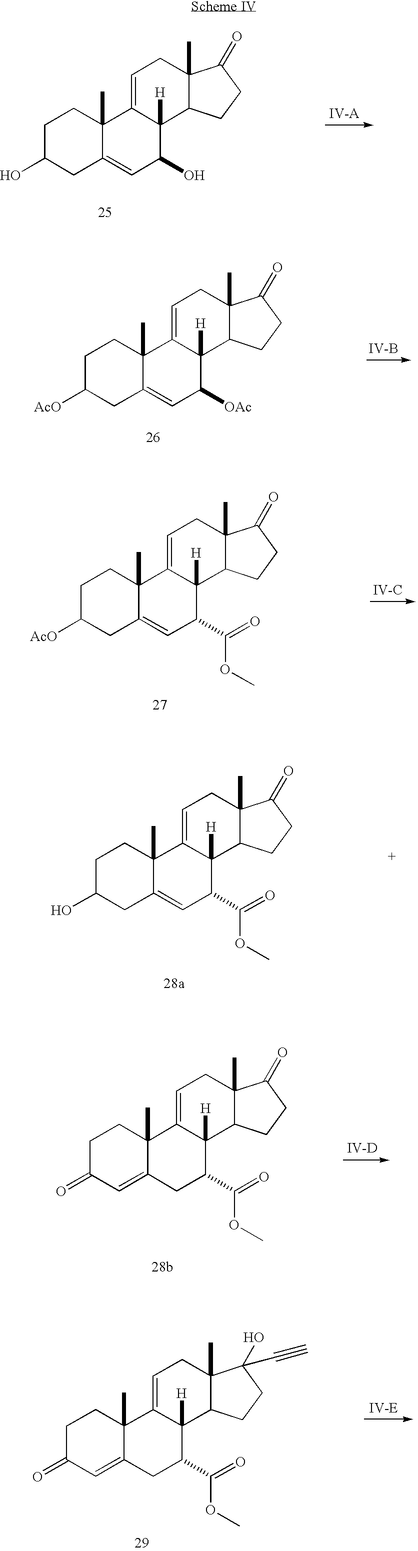

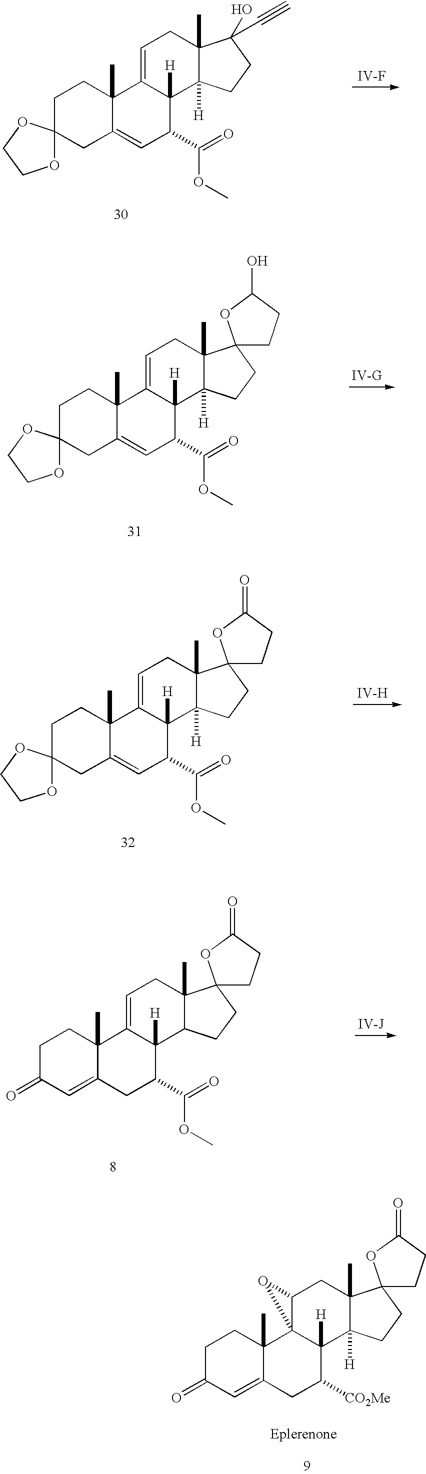

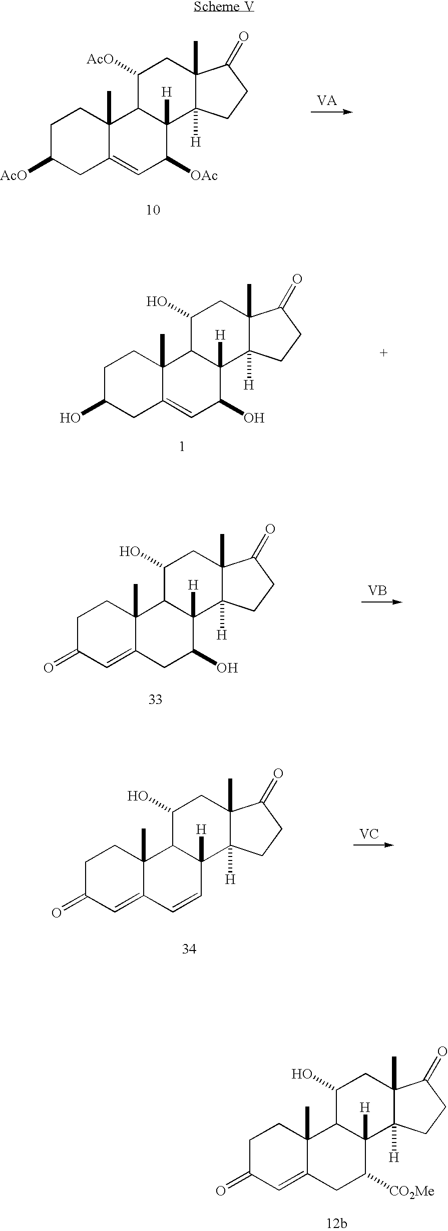

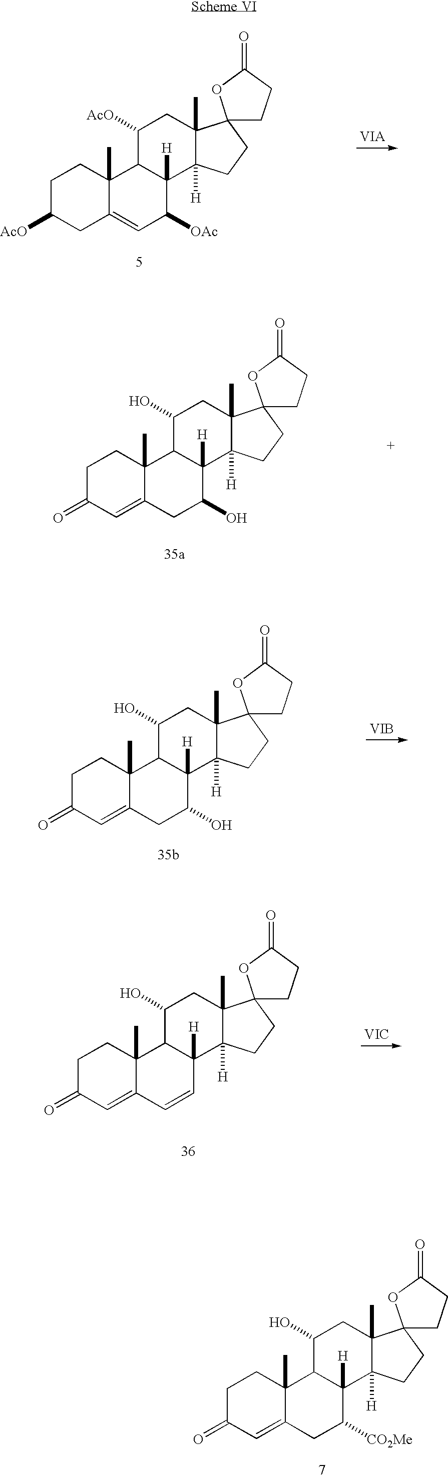

Preparation of the starting material 1, (3β,7β,11α-trihydroxy-5-androsten-17-one) for Schemes I-II is obtained in one of two ways. One way is to first contact 5-anrosten-3β-ol-17-one with a submerged culture of Diplodia gossypina ATCC 20517 (synonym Botryodiplodia theobromae IFO 6469) to generate 5-androsten-3β,7β-diol-17-one (see Example 10), and then contact 5-androsten-3β,7β-diol-17-one with a submerged culture of Aspergillus ochraceus ATCC 18500 to generate 5-androsten-3β,7β,11α-triol-17-one 1. Alternatively, one can contact 5-anrosten-3β-ol-17-one with a submerged culture of Absidia coerulea ATCC 6647 to generate 5-androsten-3β,7β,11α-triol-17-one 1. The starting material for Scheme IV (25) is obtained by first contacting 5-anrosten-3β-ol-17-one with a submerged culture of Aspergillus ochraceus ATCC 18500 to generate 5-androsten-3β,11α-diol-17-one (see Example 13), then chemically eliminating the 11α-hydroxyl to generate 5,9(11)-androstadien-3β-ol-17-one, followed by contacting 5,9(11)-androstadien-3β-ol-17-one with a submerged culture of Diplodia gossypina ATCC 20517 (synonym Botryodiplodia theobromae IFO 6469) to generate 5,9(11)-androstadien-31,71-diol-17-one 25.

A general description of the various steps in the processes follows.

Biotransformation of 3,11-diacyloxy-5-ene steroids to 11-hydroxy-4-ene-3-one steroids (Steps I-F, II-C, III-B, IV-C, VA and VIA): Biotransformations are accomplished as described above.

Steps I-A, II-D, III-C and IV-E: addition of acetylene to 17-oxo intermediates: 17-oxo intermediates are reacted with acetylene to provide the corresponding addition compounds according to procedures described in the literature (see for example: Schwede, W., et al., Steroids, 63 166 (1998); Corey, E. J., et al., J. Amer. Chem. Soc. (1999), 121, 710-714; Schwede, W. et al., Steroids (1998), 63(3), 166-177; Ali, H. et al., J. Med. Chem. (1993), 36(21), 3061; Turuta, A. M., et al., Mendeleev Commun. (1992), 47-8; Kumar, V. et. al., Tetrahedron (1991), 47(28), 5099; Page, P. C., Tetrahedron (1991), 47, 2871-8; Curts, S W., et al., Steroids (1991), 56, 8; Kataoka, H. et al., Chem. Lett. (1990), 1705-8; Christiansen, R. G. et al., J. Med. Chem. (1990), 33(8), 2094-2100).

Steps I-B, II-A and IVA: Hydroxy Acylations

Hydroxy intermediates are acylated with an acylating reagent in the presence of a tertiary organic base by procedures well known in the art. Acylating reagents include lower alkanoic anhydrides, lower alkanoic chlorides and the like. Suitable tertiary organic bases include pyridine, 4-dimethyaminopyridine, 4-dimethyaminopyridine N-oxide, triethyl amine, diisopropylethyl amine and the like.

Steps I-C, II-F, III-E and IV-F: Hydroformylation of acetylene adducts Formation of the lactol intermediates is achieved by hydroformylation with carbon monoxide and hydrogen in the presence of a catalytic amount of rhodium catalyst and a rhodium coordinating ligand according to procedures described in the literature (Wuts, P. G. M., et al., J. Org. Chem. 1989, 54, 5180; Botteghi, C., et al., Tetrahedron, 2001, 57, 1631). The reaction is conducted at a pressure of from 14-500 psi, preferably from 100-200 psi. The ratio of hydrogen to carbon monoxide is 1/5 to 5/1, preferably 1/1. Suitable rhodium catalysts include rhodium acetate, rhodium chloride, hydridorhodiumtristriphenylphosphine and dicarbonyl acetylacetonato rhodium II. Suitable ligands include triarylphosphines, trialkyl phosphites bidentate phosphines such as xantphos, bidentate phosphites and the like.

Steps I-D, II-G and III-F: Oxidation of Lactols to Lactones:

Oxidation of lactols to lactones can be achieved with a variety of standard oxidizing reagents. Examples of suitable oxidizing reagents include: Iodosuccinimide/tetrabutyl ammonium iodide (Kraus, G. A., et al., Bioorganic & Medicinal Chemistry Letters (2000), 10(9), 895-897; Barrett, A. G. M., et al., J. Org. Chem. (1989), 54(14), 3321); Jones reagent (chromic acid in acetone) (Panda, J., et al., Tetrahedron Letters (1999), 40, 6693; Tomioka, K., et al., J. Org. Chem. (1988), 53(17), 4094; Silver carbonate (Chow, T. J., et al., J. Chem. Soc., Perkin Transactions 1, (1999), 1847); Pyridinium chlorochromate (Uchiyama, M., et al., Tetrahedron Letters (2000), 41(51), 10013; Vanderiei, J. M. de L., Synthetic Communications (1998), 28(16), 3047; Kassou, M., et al., J. Org. Chem. (1997), 62, 3696; Rehnberg, N., et al., J. Org. Chem. (1990), 55(14), 4340-9; RuO₄/tetralkylammonium salts/tert-amine N-oxide, Jeewoo, K., et al., Chem. Lett. (1995), (4), 299; pyridinium dichromate, Paquette, L. A., et al., J. Am. Chem. Soc. (1995), 117(4), 1455-6); sodium hypochlorite/tert-amine N-oxide (Waldemar, A., et al., Chem. Rev., (2001), 101, 3499); aluminum alkoxides/acetone (Ooi, T., et. al., Synthesis (2002), 279; Djerassi, C., et al., Org. React. (1951), 6, 207); triacetoxyperiodoindane (Martin, J. C., et al., J. Amer. Chem. Soc., (1991), 113, 7277).

Steps I-E, II-B and IV-B: Carbonylation at C-7

Carbonylation of steroidal Δ⁵-ene-7-acylates (Compounds 5, 10 and 26) is accomplished by reaction with carbon monoxide in the presence of an alcohol, a base, a palladium catalyst and, optionally, a co-solvent, to provide the steroid compounds of Formula I according to methods described in the literature (Tsuji, J., et al., J. Org. Chem., (1984), 49, 1341; Murahashi, S.-I., et al., J. Org. Chem., (1993), 58, 1538; Satoh, T., et al., J. Org. Chem., (1997), 62, 2662; Cao, P., et al., J. Amer. Chem. Soc., (1999) 121, 7708; Brunner, M., et al., J. Org. Chem., (1997), 62, 7565; Gabriele, B., J. Mol. Catal., (1996), 111, 43; Yamamoto, A., et al., Helv. Chim. Acta, (2001), 84, 2996). Suitable palladium catalysts include, but are not limited to, palladium acetate, palladium(II) acetylacetonate, palladium(0)bis(dibenzylideneacetone) (Pd₂(dba)₂), palladium 1,3-diphenylphosphinopropane dibromide, (Pd(dppp)Br₂), dimethyl-2-(dimethylphosphino)ethylphosphine palladium and bistriphenylphosphine palladium dibromide (Pd₂(Ph₃P)₂Br₂. Suitable bases include, but are not limited to N-methylmorpholine (NMM), triethylamine (TEA), diisopropylethylamine (DIPEA) and the like. Reactions were conducted at 70-80° C. and 1200-1400 psi carbon monoxide in methanol for 10-12 hrs. The reaction mixture optionally contains bromide from, for example, lithium bromide. The results of carbonylation under a variety of conditions are summarized in Table 1. As can be seen, yields of product are dependent on conditions and range from 0% up to nearly 80%. Specific conditions for this reaction are found in the examples.

Steps I-G, II-I and III-H: Dehydration of 11hydroxy Intermediates:

Dehydration of 11-hydroxy intermediates 7b and 18b is achieved using phosphorous pentachloride as has been described (U.S. Pat. No. 4,559,332). Alternatively, the 11-hydroxy intermediates may be converted to a sulfonyl ester, for example a methane sulfonate or a p-toluene sulfonate, followed by treatment with a base to affect elimination as is described in WO97/21720 and WO98/25948.

Step III-A: Allylation of 2-methylfuran;

Reaction of the triacylated compound 10 with 2-methylfuran in the presence of a Lewis acid, usually in an inert solvent such as acetonitrile or methylene chloride, gives 17. Suitable Lewis acids include, but are not limited to, transition element triflates (OTf=OSO₂CF₃) such as Sc(OTf)₃, Ce(OTf)₃, and Yb(OTf)₃, and Molybdenum(II) complexes such as Mo(CO)₅(OTf)₂ and [Mo(CO)₄Br₂]₂.

Step III-I: Conversion of 7-furanyl steroids to 7 carbomethoxy steroids:

Degradation of the furan ring in 24 to the methyl ester 8 is achieved by ozonolysis, oxidation and esterification as described in the examples.

Steps I-H, II-Hand III-H: Oxidation of C-9,11 Olefins to Epoxides:

Methods for conversion of the known intermediate 8 to 9 (eplereneone) are described in U.S. Pat. Nos. 3,095,412, 4,559,332, and 5,981,744.

Steps VB and VIB: Dehydration of 7-hydroxy-4-ene-3-one Steroids to 4,6-diene-3-one Steroids:

Compounds 33 and 35b are converted to the 4,6 dienes by treatment with acid in the presence of trimethylorthoformate as described in U.S. Pat. No. 4,565,657.

Steps VC and VIC: conversion of 4,6-diene-3-one Steroids to 7-carboxy-4-ene-3-one Steroids:

Dieneones 34 and 36 are converted to the corresponding 7-carbomethoxy compounds 12b and 7b by: a) treatment of the dieneone with acetone cyanohydrin in dimethylformamide in the presence of lithium chloride and triethyl amine at 85° C. for 8-15 hours; b) treatment of the product of step a) with hydrochloric acid in methanol/water at 80° C. for 5 hours; and, c) treating the product of step b) with sodium methoxide in methanol at reflux for 20 hours as described in U.S. Pat. No. 5,981,744.

### EXAMPLES

Without further elaboration, it is believed that one skilled in the art can, using the preceding descriptions, practice the present invention to its fullest extent. The following detailed examples describe how to prepare the various compounds and perform the various processes of the invention and are to be construed as merely illustrative, and not limitations of the preceding disclosure in any way whatsoever. Those skilled in the art will promptly recognize variations from the procedures both as to reactants and as to reaction conditions and techniques.

### Example 1

### Biotransformation of 5-androsten-3β,7β,11α-triacetoxy-17-one 10 to 4-androsten-7β,11α-diol-3,17-dione 27 and/or 5-androsten-3β,7β,11α-triol-17-one 1 is performed using a submerged culture of Flavobacterium dehydrogenans ATCC 13930.

### (A) Primary-Seed Stage

Frozen vegetative cells of Flavobacteriun dehydrogenans ATCC 13930 are thawed, transferred to nutrient agar (Difco) plates, and incubated at 28° C. for 72 hours. A single colony of Flavobacterium dehydrogenans ATCC 13930 is used to inoculate a 500-mL shake flask containing 100 mL primary-seed medium. Primary-seed medium consists of (per liter of RO water): nutrient broth, 8 g; glycerol, 4 mL; water-soluble brewers yeast extract, 1 g; KH₂PO₄, 2.72 g; polyoxyethylenesorbitan monooleate, 2 mL; pre-sterilization pH 6.8, adjusted with 2N NaOH. Shake flasks, containing 100 mL primary-seed medium, are sterilized for 30 minutes at 121° C. using an autoclave. Flavobacterium dehydrogenans ATCC 13930 is incubated for 48 hours at 28° C., using a controlled-environment incubator-shaker set at 270 r.p.m. (2″ orbital stroke).

(B) Secondary-Seed Stage

One hundred milliliter secondary-seed medium, in a 500-mL shake flask, is inoculated using 0.12 mL of vegetative primary-seed culture (approximately 0.12% [v/v] inoculation rate). Secondary-seed medium contains (per liter of RO water): cerelose, 20 g; hydrolyzed soy protein, 6 g; water-soluble brewers yeast extract; 6 g; silicone defoamer (SAG 471), 0.5 mL; pre-sterilization pH 6.8, adjusted with 2N NaOH. Shake flasks, containing 100 mL secondary-seed medium, are sterilized for 30 minutes at 121° C. using an autoclave. Flavobacterium dehydrogenans ATCC 13930 is incubated for 48 hours at 28° C., using a controlled-environment incubator-shaker set at 270 r.p.m. (2″ orbital stroke).

### (C) Steroid Bioconversion

Fifty milliliter steroid-bioconversion medium, in a 500-mL shake flask, is inoculated using 2.5 mL of vegetative secondary-seed culture (approximately 5% [v/v] inoculation rate). Steroid-bioconversion medium contains (per liter of water): cerelose, 5 g; water-soluble brewers yeast extract, 15 g; polyoxyethylenesorbitan monooleate, 0.1 mL; (NH₄)₂SO₄, 1 g; KH₂PO₄, 1 g; pre-sterilization pH 6.8, adjusted with 2N NaOH. Prior to sterilizing the steroid-bioconversion medium, steroid substrate dissolved in a minimal volume of acetone is added to vigorously stirring medium to a final concentration of 1 g/L. Shake flasks, containing 50 mL steroid-bioconversion medium, are sterilized for 30 minutes at 121° C. using an autoclave. Flavobacterium dehydrogenans ATCC 13930 is incubated at 28° C., using a controlled-environment incubator-shaker set at 270 r.p.m. (2″ orbital stroke) for 96 hours. The progress of the biotransformation was followed by thin layer chromatography using Analtech silica gel plates developed with cyclohexane: ethylacetate: methanol: glacial acetic acid (90:60:30:1, v/v/v/v).

### (D) Isolation Procedure

Three hundred and fifty milliliters of whole beer at harvest, from seven shake flasks (initial substrate charge 350 mg) is extracted with an equal volume of methylene chloride for one hour. This operation is repeated to maximize product recovery. The organic extracts are separated from the spent aqueous by centrifugation. The methylene chloride extracts are polished, pooled, dried onto 5 g of silica gel G-60 by distillation, and placed on top of 100 g of silica gel G-60 in a 1″×20″ glass column equilibrated with 95% methylene chloride and 5% methanol. The chromatography is developed with the same 95% methylene chloride and 5% methanol mixture. The column eluate is collected in 20 mL fractions and the development monitored by TLC using the same 95% methylene chloride and 5% methanol mobile phase. Fractions of each of the two end products are combined and each is concentrated by evaporation to about 5-10 mL. About 10 mL of n-butyl acetate is added to the two concentrates. Continued concentration, and subsequent cooling to 4° C., results in product crystallization. The crystals are recovered by filtration, washed with cold n-butyl acetate and dried to give 53 mg 4-androsten-7β,11α-diol-3,17-dione, 27 and 18 mg 5-androsten-3β,7β,11α-triol-17-one, 1.

### Example 2

### Bioconversion of 5-androsten-3β,11α-diacetoxy-7α-carbomethoxy-17-one 11 to 4-androsten-11α-ol-7α-carbomethoxy-3,17-dione 12b and/or 5-androsten-3β,11α-diol-7α-carbomethoxy-17-one 12a is performed using a submerged culture of Flavobacterium dehydrogenans ATCC 13930.

Under the conditions described in EXAMPLE 1, but using one liter of whole beer at harvest from 20 shake flasks (initial substrate charge 1 g), 611 mg of 5-androsten-3β,11α-dihydroxy-7α-carbomethoxy-17-one, 12b, and 49 mg 4-androsten-11α-ol-7α-carbomethoxy-3,17-one, 12a, are made.

### Example 3

### Bioconversion of pregn-5-ene-7α,21-dicarboxylic acid-3β,11α-diacetoxy-17β-hydroxy-γ-lactone methyl ester 6 to pregn-4-ene-7α,21-dicarboxylic acid-3-oxo-11α,17β-dihydroxy-γ-lactone methyl ester, 7b and pregn-5-ene-7α,21-dicarboxylic acid-3β,11α,17β-trihydroxy-γ-lactone methyl ester 7a is performed using a submerged culture of Flavobacterium dehydrogenans ATCC 13930.

Under the conditions described in EXAMPLE 1, but using 1.6 liters of whole beer at harvest from 32 shake flasks and a substrate charge of 1.6 g, 216 mg pregn-4-ene-7α,21-dicarboxylic acid-3-oxo-11α,17β-dihydroxy-γ-lactone methyl ester, 7, and 767 mg pregn-5-ene-7α,21-dicarboxylic acid-3β,11α,17β-trihydroxy-γ-lactone methyl ester are made.

### Example 4

### Bioconversion of 5-androsten-3β,11α-diacetoxy-7β-furan-17-one 15 to 5-androsten-3β,11α-diol-7α-furan-17-one 16a and 4-androsten-11α-ol-7α-furan-3,17-dione 16b is performed using a submerged culture of Flavobacterium dehydrogenans ATCC 13930.

Under the conditions described in EXAMPLE 1, but using one liter of whole beer at harvest from 15 shake flasks (initial substrate charge 1 g), 126 mg of 5-androsten-3β,11α-dihydroxy-7α-furan-17-one 16a and 97 mg 4-androsten-11α-ol-7α-furan-3,17-one 16b are made.

### Example 5

### Bioconversion of 3β,7β,11α-triacetoxy-17β-hydroxypregn-5-ene-21-carboxylic acid γ-lactone 5 to 7β,11α, 17β-trihydroxy-3-oxo-pregn-4-ene-21-carboxylic acid, γ-lactone 35a, 7α,11α, 17β-trihydroxy-3-oxo-pregn-4-ene-21-carboxylic acid, γ-lactone 35b and 11α,17β-dihydroxy-3-oxo-pregn-4,6-diene-21-carboxylic acid, γ-lactone 36 is performed using a submerged culture of Flavobacterium dehydrogenans ATCC 13930.

Under the conditions described in EXAMPLE 1, but using one liter of whole beer at harvest from 15 shake flasks (initial substrate charge/flask 1 g), 30 mg of 7β,11α, 17β-trihydroxy-3-oxo-pregn-4-ene-21-carboxylic acid, γ-lactone 35a, 95 mg 7α,11α, 17β-trihydroxy-3-oxo-pregn-4-ene-21-carboxylic acid, γ-lactone 35b, and 20 mg 11α,17β-dihydroxy-3-oxo-pregn-4,6-diene-21-carboxylic acid, γ-lactone 36 are made.

### Example 6

Addition of acetylene to 17-oxo intermediates:

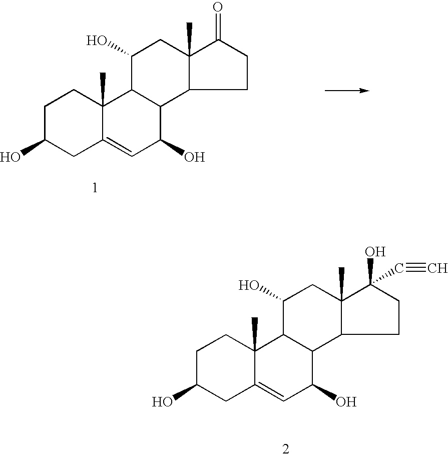

Hexamethyldisilazane (HMDS) (100 ml) is added to a stirred slurry of 50.0 g Triol 1 in 400 ml methylene chloride. Saccharin (0.57 g) is added and the mixture is heated under reflux for 3 hours during which time the slurry will gradually dissolve to a clear, amber solution. Water (5 ml) is added to quench any excess HMDS. After 5 minutes at reflux the mixture is filtered through a CH₂Cl₂ wet layer of 32.6 g magnesol on a 350 ml coarse frit filter funnel. The filtrate should be clear and almost colorless. The filter cake is washed with 2×100 ml CH₂Cl₂. The combined filtrates are concentrated under reduced pressure and residual methylene chloride is removed by evaporation with 2×500 ml portions of tetrahydrofuran (THF), concentrating to dryness after each addition to give a white solid.

A suspension of potassium t-butoxide (42.0 g) in 500 ml THF is cooled to 9°±5° C. with an ice/methanol bath. Acetylene is bubbled into the mixture just under the surface with moderate stirring at for at least 1 hour. The silylated steroid intermediate from above in THF (400 ml) is added over 30 minutes while maintaining a reaction temperature of 0°±5° C. After the addition, the mixture is stirred for a further hour at 5°±5° C. Water (100 ml) is added slowly allowing the reaction mixture to warm up to 150±5° C. 125 ml of 10% HCl is slowly added to reduce the pH to 2.5 to 3. The mixture is stirred at pH 2.5 to 3, adding small amounts of 5% HCl as needed to maintain a pH of 2.5 to 3, for 1 to 2 hours at 20°±5° C. When the hydrolysis is complete, half saturated NaHCO₃ solution is added to raise the pH to 5.5 to 6. The mixture is diluted with ethyl acetate (500 ml) and the phases separated. The aqueous phase is extracted with ethyl acetate and the combined ethyl acetate phases are washed with water, brine, dried over magnesium sulfate and concentrated to give the addition product 2.

### Example 7

Hydroxy Acetylations

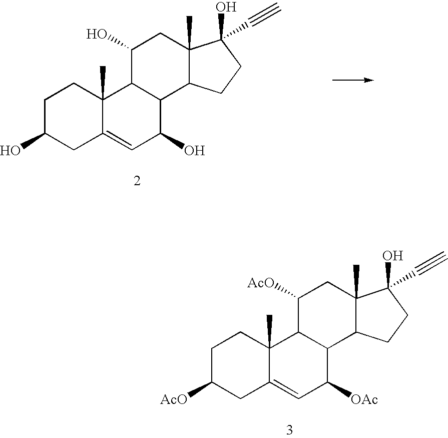

A mixture of the tetraol 2 (50.00 g, 144 mmol) dissolved in pyridine (150 ml) is cooled to <10° C. in an ice bath. Dimethylaminopyridine (DMAP) (1.7 g, 14 mmol) is added followed by slow addition of acetic anhydride (41.4 ml, 439 mmol) at a rate to maintain the solution temperature below 10° C. Following the addition, the reaction mixture is warmed to room temperature. The mixture is diluted with ethyl acetate (75 ml) and water (50 ml), stirred for 5 minutes and the layers separated. The organic layer is washed with 10% HCl (4×25 ml) followed by H₂O (2×50 ml), dried over MgSO₄ and concentrated. The product is recrystallized from toluene (100 ml).

### Example 8

Hydroformylation of Acetylene Adducts

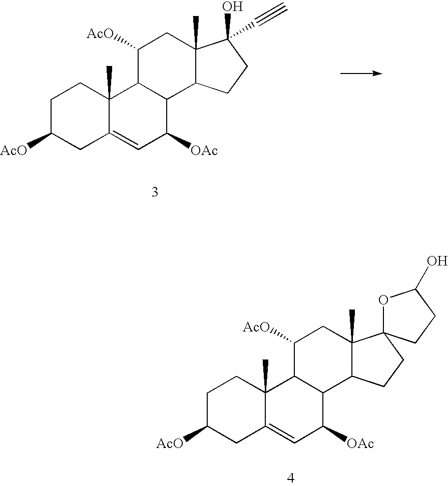

A solution of the triacetate 3 (25.4 g, 54 mmol), PPh₃ (2.13 g, 8.1 mmol) and Rh₂(OAc)₄ (716 mg, 1.62 mmol) in ethyl acetate (200 ml) is heated at 80° C. under a 1/1 mixture of hydrogen/carbon monoxide at a pressure of 170 psi for 12 hours. The mixture is concentrated under reduced pressure and the product 4 purified by column chromatography (70/30 EtOAc/Hex and 500 g silica).

### Example 9

Oxidation of Lactols to Lactones

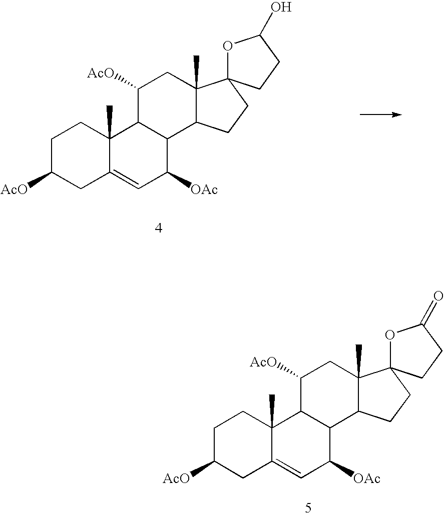

A mixture of the lactol 4 (25 g, 50 mmol), methylene chloride (250 ml), water (38 ml), 2,2,6,6-tetramethylpiperidin-1-oxyl (TEMPO) (156 mg, 1 mmol), KBr (595 mg, 5 mmol), and NaHCO₃ (5.5 g, 65 mmol) is cooled to <10° C. in an ice bath. A solution of 1.1 M sodium hypochlorite (NaOCl) (50 ml, 55 mmol) is slowly added. The mixture is allowed to warm to room temperature and diluted with water (50 ml). The layers are separated and the organic layer washed with brine (2×50 ml). The organic layer is dried with MgSO₄, filtered and concentrated to afford 5 as an off white foam.

### Example 10

Carbonylation at C-7

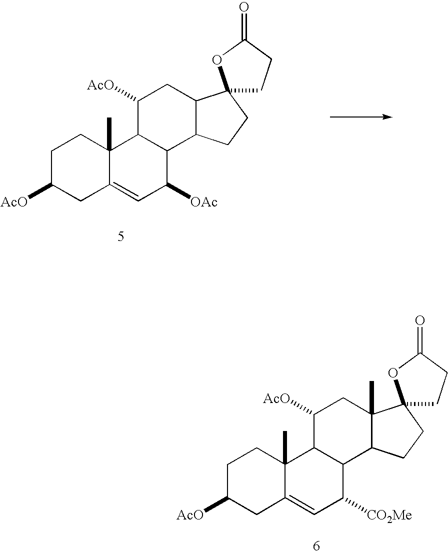

The triacetate 5 (2.0 g), Pd(dppp)Br₂ (126 mg), diisopropyl amine (0.78 mL), Et₄NBr (260 mg), NaBr (1.09 g) in 20 ml of methanol is pressurized to 1200 psi with CO then heated at 65° C. for twelve hours. The solution is cooled and concentrated and the residue chromatographed on silica gel with 40-75% ethyl acetate/hexane to give the methyl ester 6.

### Example 11

Dehydration of 11-hydroxy Intermediates

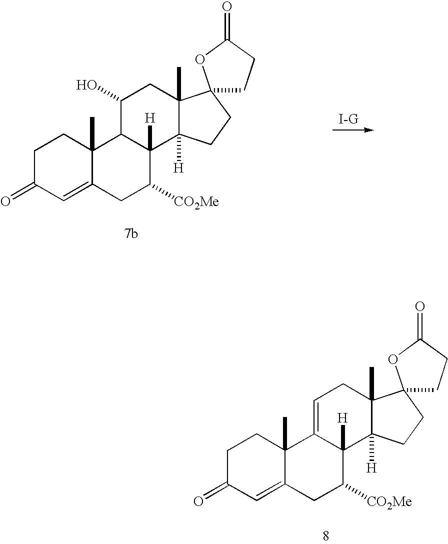

Phosphorous pentachloride (2 eq) is added to a solution of the alcohol 7 (1 eq) in THF at −51° C. which results in a temperature rise to −48° C. After 2 hours the mixture is poured into aqueous NaHCO₃ and extracted with EtOAc and concentrated. The residue is chromatographed on silica gel with EtOAc/hexane to afford the diene 8.

Preparation of 5-androsten-3β,7β-diol-17-one to 5-androsten-3β,7β,11α-triol-17-one, starting material 1.

Step 1

### Example 12

### Bioconversion of 5-androsten-3β-ol-17-one to 5-androsten-3β,7β-diol-17-one

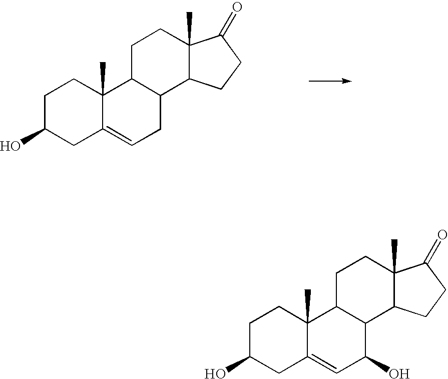

The bioconversion of 5-androsten-3β-ol-17-one to 5-androsten-3β,7β-diol-17-one is performed using a submerged culture of Diplodia gossypina ATCC 20571 (synonym Botryodiplodia theobromae IFO 6469) at a 10-L fermentation scale.

### (A) Primary-Seed Stage

Frozen vegetative cells of Diplodia gossypina ATCC 20571 are thawed, transferred to potato-dextrose-agar plates (PDA), and incubated at 28° for 72 hours. Single mycelial-plugs (6-7 mm diam.) are used to inoculate siliconized 500-mL stippled shakeflasks containing 100 mL primary-seed medium. Primary-seed medium consists of (per liter of RO water): dextrin, 50 g; soyflour, 35 g; cerelose, 5 g; cobalt chloride hexahydrate, 2 mg; silicone defoamer (SAG 471), 0.5 mL; pre-sterilization pH 7.0-7.2, adjusted with sodium hydroxide (2N). Diplodia gossypina ATCC 20571 is incubated for 48 hours at 28°, using a controlled-environment incubator-shaker set at 280 r.p.m. (1″ orbital stroke).

### (B) Secondary-Seed Stage

Ten-liter secondary-seed fermentations are inoculated using 1.2 mL vegetative primary-seed culture (0.012% [v/v] inoculation rate). Secondary-seed medium contains (per liter of RO water): cerelose, 60 g; soyflour, 25 g; soybean oil, 30 mL; magnesium sulfate heptahydrate, 1 g; potassium dihydrogen phosphate, 0.74 g; polyoxyethylenesorbitan monooleate, 2 mL; silicone defoamer (SAG 471), 0.5 mL; pre-sterilization pH 3.95-4.00, adjusted with concentrated sulfuric acid. The fermentors, containing secondary-seed medium, are sterilized for 20 minutes at 121° using both jacket and injection steam. The agitation rate during sterilization is 200 r.p.m. Post-sterilization, the medium pH is adjusted to 4.0 using sterile sulfuric acid (5%). Diplodia gossypina ATCC 20571 is incubated at 28° using the following initial parameters: agitation, 100 r.p.m.; back pressure=5 psig; airflow=2.5 SLM (0.25 VVM); low DO set-point, 30%; pH control, none. When the DO first drops to 30%, the airflow is increased to 5 SLM (0.5 VVM). When the culture reaches low DO again, 30% DO is maintained using agitation control. Secondary-seed cultures are harvested at approximately 60 hours post-inoculation, when the OUR is between about 10 and about 15 mM/L/h.

### (C) Steroid Bioconversion

Ten-liter steroid-bioconversion fermentations are inoculated using 500 mL vegetative secondary-seed culture (5% [v/v] inoculation rate). Steroid-bioconversion medium is the same as secondary-seed medium. Sterilization conditions and pH adjustment are as described for secondary-seed medium. Diplodia gossypina ATCC 20571 is incubated at 28° using essentially the same initial parameters as those used for secondary-seed cultivation, with the exception that the low DO set-point is increased from 30% to 50%. When the DO first drops to 50%, the air flow is increased from 2.5 SLM (0.25 VVM) to 5 SLM (0.5 VVM). When the culture reaches low DO again, 50% DO is maintained using agitation control. Starting at 24 hours post-inoculation, micronized 5-androsten-3β-ol-17-one, slurried in a minimal volume of 0.2% polyoxyethylenesorbitan monooleate, is added to the fermentation in one-hour intervals until 400 g total is added. At about 3 days post-inoculation, an additional 100 g cerelose is added to the 10-L fermentation.

Bioconversion cultures are assayed on a daily basis for 5-androsten-3β,7β-diol-17-one using TLC. One milliliter of whole beer is extracted with 10 mL methanol. Cells are separated from the aqueous-methanol mixture by centrifugation (3,000×g for 10 minutes), and several microliters applied to a TLC plate. The TLC plate is developed in cyclohexane:ethyl acetate:methanol (90:60:15) and the product visualized by spraying the TLC with 50% sulfuric acid, followed by charring in an oven. Product is compared with authentic standard, which turns blue on spraying with 50% sulfuric acid. Bioconversion of 5-androsten-3β-ol-17-one to 5-androsten-3β,7β-diol-17-one is complete approximately 4 days post-inoculation.

### (D) Isolation Procedure

The whole beer at harvest is centrifuged and the rich solids are recovered by centrifugation. The rich solids are extracted with 10 liters of methylene chloride and the rich extract is recovered by centrifugation. The extract is polished and concentrated to about 1-liter by distillation and the crystal slurry is cooled to −10° C. The crystals are recovered by filtration, washed with cold methylene chloride to remove color, and dried to give 227 grams of purified crystalline 5-androsten-3β,7β-diol-17-one.

Step 2

### Example 13

### Bioconversion of to 5-androsten-3β,7β-diol-17-one to 5-androsten-3β,7β,11α-triol-17-one.

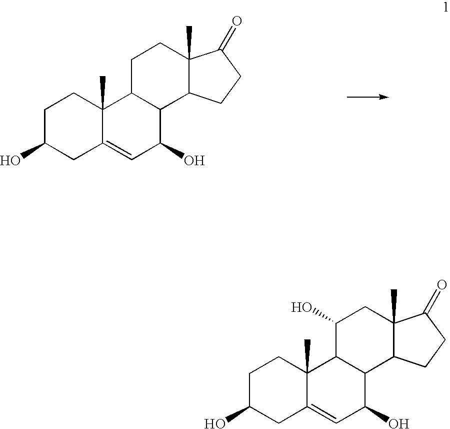

The bioconversion of 5-androsten-3β,7β-diol-17-one to 5-androsten-3β,7β,11α-triol-17-one is performed using a submerged culture of Aspergillus ochraceus ATCC 18500 (synonym NRRL 405) at a 10-L fermentation scale.

### (A) Primary-Seed Stage

Primary-seed cultures of Aspergillus ochraceus ATCC 18500 are prepared as described for Diplodia gossypina ATCC 20571 in EXAMPLE 12.

### (B) Secondary-Seed Stage

Ten-liter secondary-seed fermentations are inoculated using 1.2 mL vegetative primary-seed culture (0.012% [v/v] inoculation rate). Secondary-seed medium contains (per liter of RO water): cerelose, 40 g; soyflour, 25 g; soybean oil, 30 mL; magnesium sulfate heptahydrate, 1 g; potassium dihydrogen phosphate, 0.74 g; nonylphenoxypolyethoxyethanol, 0.25 mL; silicone defoamer (SAG 471), 0.5 mL; pre-sterilization pH 3.95-4.00, adjusted with concentrated sulfuric acid. The fermentors, containing secondary-seed medium, are sterilized for 20 minutes at 121° using both jacket and injection steam. The agitation rate during sterilization is 200 r.p.m. Post-sterilization, the medium pH is adjusted to 4.0 using sterile sulfuric acid (5%). Aspergillus ochraceus ATCC 18500 is incubated at 28° using the following initial parameters: agitation, 100 r.p.m.; back pressure=5 psig; airflow=2.5 SLM (0.25 VVM); low DO set-point, 50%; pH control, none. When the DO first drops to 50%, the airflow is increased to 5 SLM (0.5 VVM). When the culture reaches low DO again, 50% DO is maintained using agitation control. Secondary-seed cultures are harvested between 50 to 54 hours post-inoculation, when the OUR is between about 20 and about 26 mM/L/h.

### (C) Steroid Bioconversion

Ten-liter steroid-bioconversion fermentations are inoculated using 500 mL vegetative secondary-seed culture (5% [v/v] inoculation rate). Steroid-bioconversion medium is essentially the same as secondary-seed medium, with the exception that the nonylphenoxypolyethoxyethanol is increased from 0.25 mL/L to 2 mL/L, and pre-sterilization pH is adjusted to 2.95-3.00 with concentrated sulfuric acid. Sterilization conditions are as described for secondary-seed medium. Post-sterilization, the medium pH is adjusted to 3.0 using sterile sulfuric acid (5%). Aspergillus ochraceus ATCC 18500 is incubated at 28° using essentially the same initial parameters as those used for secondary-seed cultivation, with the exception that agitation is initially set at 200 r.p.m. At about 18 hours post-inoculation, 200 g micronized 5-androsten-3β,7β-diol-17-one, slurried in a minimal volume of 0.2% nonylphenoxypolyethoxyethanol, is added to the 10-L fermentation.

Bioconversion cultures are assayed on a daily basis for 5-androsten-3β,7β,11α-triol-17-one using TLC, as described in EXAMPLE 10. Bioconversion of 5-androsten-3β,7β-diol-17-one to 5-androsten-3β,7β,11α-triol-17-one is complete approximately 4 days post-inoculation.

### (D) Isolation Procedure

The whole beer solids are recovered by centrifugation. The liquid is discarded. The rich solids are extracted with 10 liters of 80% acetone 20% water at 45° C. to 50° C. and the warm extract is clarified by filtration. The rich filtrate is concentrated by distillation to remove acetone generating an aqueous slurry of crude crystals. The crude crystals are recovered by filtration and the mother liquor is discarded. The water-wet crystals are triturated in 600 milliliters of methylene chloride to remove impurities, dissolved in 700 milliliters of methanol (by heating to 55° C.), and then decolorized with 5 grams of Darco G-60 carbon. After filtration to remove carbon, the filtrate is concentrated to crystallize the product. The methanol is removed further by adding 300 mL of n-butyl acetate and concentrating to a thick crystal slurry. The crystals are filtered, washed with n-butyl acetate, and dried to give 158 grams of purified crystalline 5-androsten-3β,7β,11α-triol-17-one.

### Example 14

### Preparation of 1, Method 2, Bioconversion of 5-androsten-3β-ol-17-one to 5-androsten-3β,7β,11α-triol-17-one.

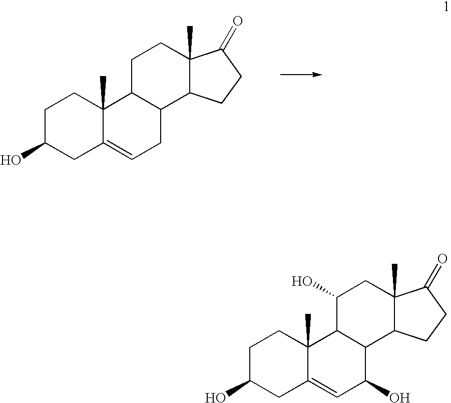

The bioconversion of 5-androsten-3β-ol-17-one to 5-androsten-3β,7β,11α-triol-17-one is performed using a submerged culture of Absidia coerulea ATCC 6647 at a 10-L fermentation scale.

### (A) Primary-Seed Stage

Primary-seed cultures of Absidia coerulea ATCC 6647 are prepared as described for Diplodia gossypina ATCC 20571 in EXAMPLE 12.

### (B) Secondary-Seed Stage

Ten-liter secondary-seed fermentations are inoculated using 1.2 mL vegetative primary-seed culture (0.012% [v/v] inoculation rate). Secondary-seed medium contains (per liter of RO water): dextrin, 50 g; soyflour, 35 g; cerelose, 5 g; cobalt chloride hexahydrate, 2 mg; silicone defoamer (SAG 471), 0.5 mL; pre-sterilization pH 4.95-5.00, adjusted with concentrated sulfuric acid. The fermentors, containing secondary-seed medium, are sterilized for 20 minutes at 121° using both jacket and injection steam. The agitation rate during sterilization is 200 r.p.m. Post-sterilization, the medium pH is adjusted to 5.0 using sterile sulfuric acid (5%). Absidia coerulea ATCC 6647 is incubated at 28° using the following initial parameters: agitation, 100 r.p.m.; back pressure=5 psig; airflow=2.5 SLM (0.25 VVM); low DO set-point, 50%; pH control, none. When the DO first drops to 30%, the airflow is increased to 5 SLM (0.5 VVM). When the culture reaches low DO again, 30% DO is maintained using agitation control. Secondary-seed cultures are harvested about 76 hours post-inoculation, when the OUR is between about 4 and about 7 mM/L/h.

### (C) Steroid Bioconversion

Ten-liter steroid-bioconversion fermentations are inoculated using 500 mL vegetative secondary-seed culture (5% [v/v] inoculation rate). Steroid-bioconversion medium contains (per liter of RO water): dextrin, 50 g; soyflour, 35 g; cerelose, 20 g; silicone defoamer (SAG 471), 0.5 mL; pre-sterilization pH 2.95-3.00, adjusted with concentrated sulfuric acid. Sterilization conditions are as described for secondary-seed medium. Post-sterilization, the medium pH is adjusted to 3.0 using sterile sulfuric acid (5%). Absidia coerulea ATCC 6647 is incubated at 28° using the same initial parameters as those used for secondary-seed cultivation. At about 17 hours post-inoculation, 200 g micronized 5-androsten-3β-ol-17-one, slurried in a minimal volume of 0.2% octylphenoxypolyethoxyethanol, is added to the 10-L fermentation.

Bioconversion cultures are assayed on a daily basis for 5-androsten-3β,7β,11α-triol-17-one using TLC, as described in EXAMPLE 1. Bioconversion of 5-androsten-3β-ol-17-one to 5-androsten-3β,7β,11α-triol-17-one is complete approximately 6-7 days post-inoculation.

### (D) Isolation Procedure

The whole beer solids are recovered by centrifugation. The liquid is discarded. The rich solids are extracted using 10 liters of 85% acetone 15% water at 45° C. to 50° C. and the warm extract is clarified by filtration. The rich filtrate is concentrated by distillation to remove acetone generating an aqueous slurry of crude crystals. The crystal slurry is filtered and the mother liquor is discarded. The water-wet crystals are triturated in 600 milliliters of methylene chloride to remove impurities, dissolved in 700 milliliters of methanol (by heating to 55° C.), and then decolorized with 5 grams of Darco G-60 carbon. After filtration to remove carbon, the filtrate is concentrated to crystallize the product. The methanol is removed further by adding 300 mL of n-butyl acetate and concentrating to a thick crystal slurry. The crystals are filtered, washed with n-butyl acetate, and dried to give 75.5 grams of crude crystalline 5-androsten-3β,7β,11α-triol-17-one.

The crude crystals are triturated in 600 milliliters of methylene chloride to remove additional impurities, dissolved in 700 milliliters of methanol (by heating to 55° C.), and then decolorized with 5 grams of Darco G-60 carbon. After filtration to remove carbon, the filtrate is concentrated to crystallize the product. The methanol is removed further by adding 300 mL of n-butyl acetate and concentrating to a thick crystal slurry. The crystals are filtered, washed with n-butyl acetate, and dried to give 42.1 grams of purified crystalline 5-androsten-3β,7β,11α-triol-17-one.

### Example 15

### Bioconversion of 5-androsten-3β-ol-17-one to 5-androsten-3β,11α-diol-17-one

The bioconversion of 5-androsten-3β-ol-17-one to 5-androsten-3β,11α-diol-17-one is performed using a submerged culture of Aspergillus ochraceus ATCC 18500 (synonym NRRL 405) at a 10-L fermentation scale.

### (A) Primary-Seed Stage

Primary-seed cultures of Aspergillus ochraceus ATCC 18500 are prepared as described in EXAMPLE 13.

### (B) Secondary-Seed Stage

Ten-liter secondary-seed cultures of Aspergillus ochraceus ATCC 18500 are prepared as described in EXAMPLE 11.

### (C) Steroid Bioconversion

Ten-liter steroid-bioconversion fermentations are inoculated using 500 mL vegetative secondary-seed culture (5% [v/v] inoculation rate). Steroid-bioconversion medium is essentially the same as secondary-seed medium, with the exception that the nonylphenoxypolyethoxyethanol is increased from 0.25 mL/L to 2 mL/L, and pre-sterilization pH is adjusted to 2.95-3.00 with concentrated sulfuric acid. Sterilization conditions are as described for secondary-seed medium. Post-sterilization, the medium pH is adjusted to 3.0 using sterile sulfuric acid (5%). Aspergillus ochraceus ATCC 18500 is incubated at 28° using essentially the same initial parameters as those used for secondary-seed cultivation, with the exception that agitation is initially set at 200 r.p.m. At about 18 hours post-inoculation, 200 g micronized 5-androsten-3β-ol-17-one, slurried in a minimal volume of 0.2% nonylphenoxypolyethoxyethanol, is added to the 10-L fermentation.

Bioconversion cultures are assayed on a daily basis for 5-androsten-3β,11α-diol-17-one using TLC. One milliliter of whole beer is extracted with 19 mL methanol. Cells are separated from the aqueous-methanol mixture by centrifugation (3,000×g for 10 minutes), and several microliters applied to a TLC plate. The TLC plate is developed in cyclohexane:ethyl acetate:methanol (90:60:15) and the product visualized by spraying the TLC with 50% sulfuric acid, followed by charring in an oven. Bioconversion of 5-androsten-3β-ol-17-one to 5-androsten-3β,11α-diol-17-one is complete approximately 3 days post-inoculation.

### (D) Isolation Procedure

The whole beer solids are recovered by centrifugation. The liquid is discarded. The rich solids are extracted with 10 liters of 85% acetone 15% water at 45° C. to 50° C. and the rich extract is recovered by centrifugation. The extract is concentrated by distillation to remove acetone to generate an aqueous slurry of crude crystals. The crude crystals are recovered by filtration and the mother liquor is discarded. The water-wet crude crystals are dissolved in 700 milliliters of methanol by heating to 55° C. and then decolorized with 5 grams of Darco G-60 carbon. After filtration to remove carbon, the filtrate is concentrated to crystallize the product. The methanol is removed further by adding 300 mL of n-butyl acetate and concentrating to a thick crystal slurry. The crystals are filtered, washed with n-butyl acetate, and dried to give 174 grams of purified crystalline 5-androsten-3β,11α-diol-17-one.

### Example 16

### Preparation of 5,9(11)-androstadien-3β-ol-17-one from 5-androsten-3β,11α-diol-17-one, starting material 25

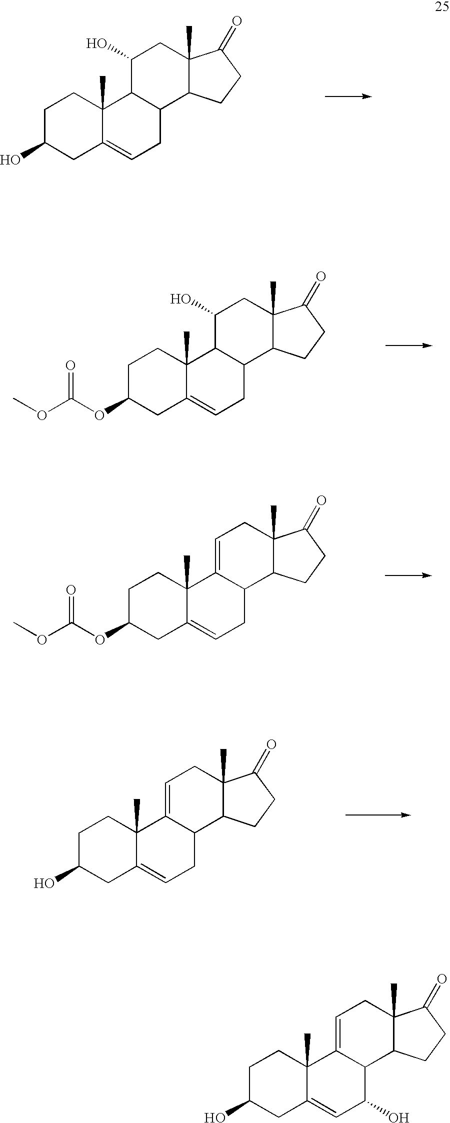

Step 1

To a slurry of 5-androsten-3β,11α-diol-17-one (30.4 g, 100 mmol) in CH₂Cl₂ (300 mL) was added TMEDA (18.1 mL, 120 mmol). The slurry was cooled to −10° C. and methyl chloroformate (7.72 mL, 100 mmol) added. The reaction was allowed to warm to room temperature. The reaction was not complete by TLC so more methyl chloroformate (772 μL, 10 mmol) was added. When the reaction was determined to be complete by TLC, EtOAc (300 mL) and H₂O (100 mL) were added and the resulting layers separated. The organic phase was washed with 50 mL H₂O, dried over MgSO₄ and concentrated to an oil which solidified on standing. The crude product was recrystallized from hot EtOAc/CH₂Cl₂ and heptane. The slurry was further cooled to 0-5° C. and the product collected by filtration (22 g, 60.8% chemical). The carbonate was further purified by column chromatography over silica gel eluting with a gradient of 5%-20% acetone/CH₂Cl₂ to obtain pure mono carbonate (20.57, 56.8%).

Step 2

The carbonate of step 1 (38.0 g, 0.105 mol) was dissolved in 570 mL of THF and cooled to −35° C. Solid PCl₅ (37.1 g, 0.178 mol) was slowly added keeping the temperature below −30° C. When TLC showed complete reaction the mixture was poured into cold NaHCO₃ solution and the product extracted with ethyl acetate. The organic layers were dried over MgSO₄ and concentrated to afford an oil.

Step 3

This oil of step 2 was dissolved in methanol (500 ml) and treated with 36.1 g of K₂CO₃ and the mixture stirred at room temperature for 15 hours. The residual carbonate was removed by filtration. The solution was partially concentrated and water added to precipitate the desired dienic alcohol, which was dried in an oven at 45° C. Yield 29.52 g

Step 4

The bioconversion of 5,9(11)-androstadien-3β-ol-17-one to 5,9(11)-androstadien-3β,7β-diol-17-one is performed using a submerged culture of Diplodia gossypina ATCC 20571 (synonym Botryodiplodia theobromae IFO 6469) at a 10-L fermentation scale.

### (A) Primary-Seed Stage

Primary-seed cultures are prepared as described in EXAMPLE 12.

### (B) Secondary-Seed Stage

Ten-liter secondary-seed cultures are prepared as described in EXAMPLE 12.

### (C) Steroid Bioconversion

Ten-liter steroid-bioconversion cultures are prepared as described in EXAMPLE 12 At about 24 hours post-inoculation, 120 g micronized 5,9(11)-androstadien-3β-ol-17-one, slurried in a minimal volume of 0.2% polyoxyethylenesorbitan monooleate, is added to the 10-L fermentation.

Bioconversion cultures are assayed on a daily basis for 5,9(11)-androstadien-3β,7β-diol-17-one using the procedure described in EXAMPLE 12. Bioconversion of 5,9(11)-androstadien-3β-ol-17-one to 5,9(11)-androstadien-3β,7β-diol-17-one is complete approximately 3 days post-inoculation.

### (D) Isolation Procedure

The rich solids from the whole beer are recovered by centrifugation. The liquid beer phase is extracted using 15 liters of methylene chloride. After settling, the upper spent beer layer is decanted and discarded. The remaining rich methylene chloride is then used to extract the rich solids. The resulting rich methylene chloride extract is drained from the spent solids, polished, concentrated by distillation to about 0.5 liters, and cooled to −10° C. The crystals obtained are recovered by filtration, washed with n-butyl acetate to remove color, and dried to give 52.2 grams of purified crystalline 5,9(11)-androstadien-3β,7β-diol-17-one.

### Example 17

### Formation of Furan 15

A solution of the triacetate 10 (2.02 mmol) in 7 mL of acetonitrile at 22° C. is treated with 2-methylfuran (0.2 mL, 2.22 mmol) and 0.298 g of Sc(OTf)₃ for 1 hour. Chromatography on silica gel with 25% EtOAc/Hex affords the furan 17.

### Example 18

### Formation of Methyl Ester 8 from Furan 20

Method A

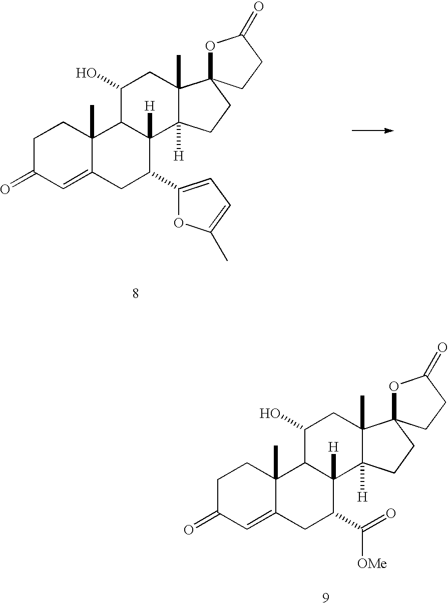

A solution of furan derivative 8 (1.0 g, 2.280 mmoles) in 100 ml methylene chloride was cooled to −79° C. A stream of O₃/O₂ was passed through the solution for 10 minutes, then the mixture was warmed to room temperature and concentrated to a solid residue, which was taken up in 50 ml 1/1 methanol/methylene chloride, treated with 1.0 ml of pyridine, and stirred at room temperature for 18 hours. The solution was then cooled to −80° C. A stream of O₃/O₂ was then passed through the solution for 4 minutes. The mixture was then diluted with 100 ml ethyl acetate and extracted with 70 ml aqueous sodium bicarbonate. The aqueous phase was acidified with aqueous hydrochloric acid to pH 0.5, then extracted with methylene chloride and concentrated to a foam (weight: 250 mg). The foam was dissolved in toluene/methanol, treated with trimethylsilyldiazomethane (0.5 ml of 2.0 M solution in hexane, 1.0 mmoles) at room temperature, then the solution was concentrated to give ester 9 as an oil.

Method B

Step 1) 5α,17β-Dihydroxypregn-9(11)-ene-3-one, 7α,21-dicarboxylic acid, bis-γ-lactone 8a.

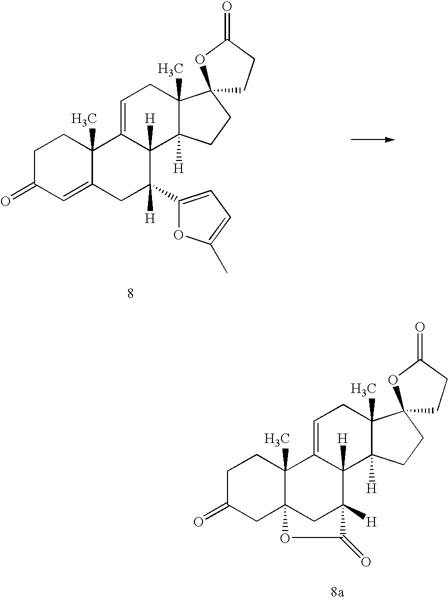

A mixture of 17β-hydroxy-7α-(5′-methyl-2′-furyl)-pregna-4,9(11)-dien-3-one-21-carboxylic acid, γ-lactone (8, 100 g, 0.23778 moles) and potassium acetate (50.0 g, 0.5094 moles, 2.14 equivalents) in acetone (500 ml) and water (150 ml) is cooled to −10° and treated with a slurry of dibromantin (34.0 g, 0.1189 moles, 0.50 molar equivalents) in water (100 ml) until a rise in the redox potential occurred. At this point, liquid chromatography analysis indicated complete conversion into a cis enedione. The reaction mixture containing the enedione is then quenched with isobutyl vinyl ether (1.0 ml, 0.768 g, 7.668 mmoles, 0.032 equivalents), concentrated to a thick slurry, diluted with methylene chloride (200 ml), and treated at 200 with concentrated hydrochloric acid (50.0 ml, 0.50 moles, 2.10 equivalents). The mixture is stirred at 20-25° for 2 hours, at which time liquid chromatography analysis indicated complete conversion to a trans enedione. The organic phase containing the enedione is separated, diluted with methylene chloride (80 ml) and methanol (300 ml), and cooled to −48°. A stream of O₃/O₂ is bubbled through this mixture until LC analysis indicated complete disappearance of the enedione (III-trans), then the mixture is quenched with dimethylsulfide (30.0 ml, 25.38 g, 0.4085 moles, 1.72 equivalents), stirred at −20° for 16 hours, concentrated to a volume of about 300 ml, diluted with methanol (350 ml), concentrated to a volume of about 300 ml, diluted with isopropanol (40 ml) and methanol (80 ml), then treated with a warm (55-60°) solution of potassium bicarbonate (120 g, 1.1986 moles, 5.04 equivalents) in water (240 ml). This slurry is cooled to 5-10°, then hydrogen peroxide (50%, 66.0 g, containing 33.0 g (0.9703 moles, 4.08 equivalents) hydrogen peroxide) is added over 3 hours. The mixture is stirred for four hours and quenched with dimethylsulfide (40 ml, 33.84 g, 0.5447 moles, 2.29 equivalents). After stirring at 20-25° for 23 hours, the mixture is diluted with methylene chloride (100 ml) and water (80 ml), and acidified to pH=3.0 with concentrated hydrochloric acid. The two-phase mixture is heated to 36°, then the phases are separated and the aqueous phase extracted with methylene chloride (100 ml). The organic phases are combined, washed with water (75 ml), and the aqueous phase is back-extracted with methylene chloride (25 ml). The organic phases are combined, concentrated to a volume of 150 ml, then treated with benzenesulfonic acid (1.0 g of 90% pure material, containing 0.90 g (5.690 mmoles, 0.0239 equivalents) benzenesulfonic acid) and acetone (50 ml). The mixture is then concentrated atmospherically to a volume of 160 ml, then diluted with acetone (250 ml), concentrated to a volume of 200 ml, cooled to 12°, and filtered. The filter cake is washed with cold acetone (2×25 ml) and dried by nitrogen stream to give the title compound, CMR (100 MHz, CDCl₃) 206.08, 176.47, 175.41, 139.63, 124.00, 94.89, 90.97, 47.08, 43.90, 42.36, 41.58, 41.07, 38.93, 36.97, 35.16, 33.01, 32.42, 32.42, 31.35, 29.10, 23.08, 22.98 and 14.23 δ; NMR (400 MHz, CDCl₃) 0.94, 1.40, 1.4-2.8 and 5.70; MS (C₁, NH₃) m/e=385 (P+H, 100%).

2) 17β-Hydroxy-7α-carbomethoxypregna-4,9(11)-dien-3-one-21-carboxylic acid, γ-lactone 9.

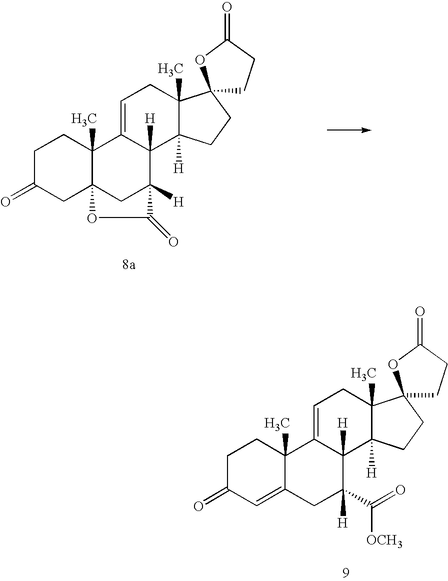

A mixture of 5α,17β-dihydroxypregn-9(11)-ene-3-one, 7α,21-dicarboxylic acid, bis-γ-lactone (8a, 50.0 g, 0.13005 moles) and potassium bicarbonate (16.92 g, 0.1690 moles, 1.30 equivalents) in acetone (200 ml) and water (100 ml) is stirred at 45° for 2 hours, at which time conversion of the 5,7-lactone (VII) into the carboxylic acid (VI) is complete by LC. The resulting mixture is then treated with dimethylsulfate (22.92 g, 0.1817 moles, 1.40 equivalents), stirred at 45° for 3 hours, then treated with a solution of potassium bicarbonate (1.3 g, 0.0130 moles, 0.100 equivalents) in water (10 ml) followed by neat triethylamine (1.81 ml, 1.314 g, 0.0130 moles, 0.100 equivalents). The mixture is stirred at 45° for 1 hour, quenched with concentrated hydrochloric acid (1.92 ml, 2.304 g, containing 0.852 g (0.0234 moles, 0.180 equivalents) hydrochloric acid), cooled to 0°, concentrated under reduced pressure to a volume of 150 ml (pot temperature 13°), then filtered and the filter cake is washed with water (2×25 ml) and dried to give the title compound 9.

### Example 19

### Formation of Eplerenone from 8

Dieneone 9 is oxidized as described in U.S. Pat. Nos. 3,095,412, 4,559,332 and 5,981,744 to give eplerenone.

What is claimed is:

1. A process for the microbial transformation of 7-substituted steroid compounds of Formula I, 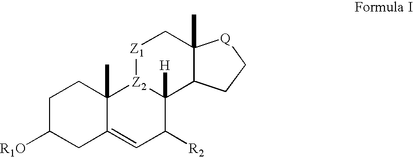
wherein:
R₁ is H or C₁-C₆-alkylC(O)—;
R₂ is O—OR, or α-C(O)—OC₁-C₆ alkyl;
Z₁ is 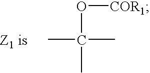
Z₂ is —CH—;
or Z₁ and Z₂ may be taken together to form a carbon-carbon double bond;
Q is 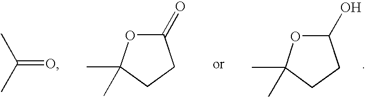
to steroid intermediates of Formula II, 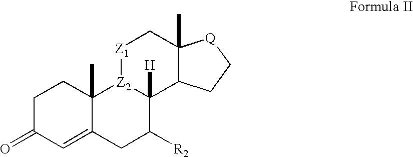
wherein R₂, Z₁, Z₂, and Q are as for Formula I;
comprising contacting a compound of Formula I with a member of the genus Flavobacterium capable of performing the transformation of a compound of Formula I to a compound of Formula II.

2. A process according to claim 1 wherein the member of the genus is Flavobacterium is selected from Flavobacterium dehydrogenans or Flavobacterium dehydrogenans, strain ATCC 13930.

3. A process according to claim 1 wherein the process is conducted in a submerged culture.

4. A process according to claim 1 for preparing eplerenone further comprising the steps:
a) biotransforming a compound of Formula 6 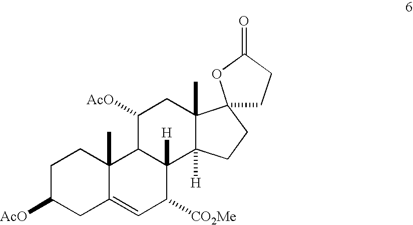
to a compound of Formula 7b; 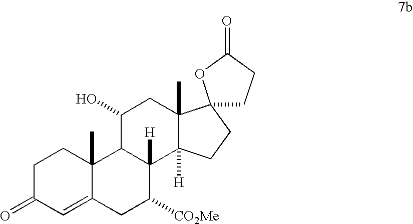
b) dehydration of a compound of Formula 7b to a compound of Formula 8; 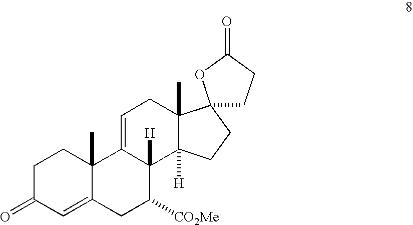
c) oxidizing a compound of Formula 8 to eplerenone, Formula 9.

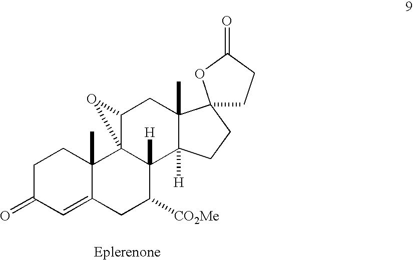

5. A process according to claim 1 for preparing eplerenone comprising the steps:
a) reacting acetylene with a compound of Formula 1 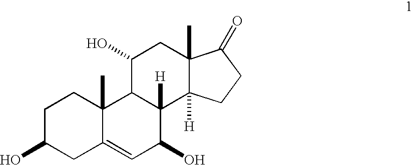
to give a compound of Formula 2; 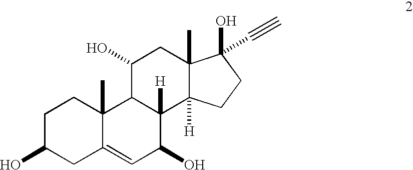
b) acetylating a compound of Formula 2 to give a compound of Formula 3; 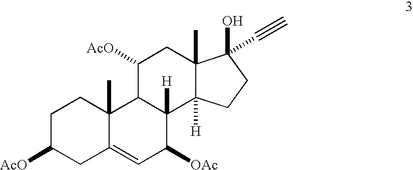
c) hydroformylating a compound of Formula 3 to give a compound of Formula 4; 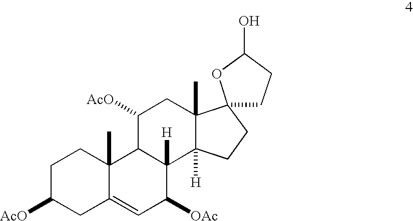
d) oxidizing a compound of Formula 4 to give a compound of Formula 5; 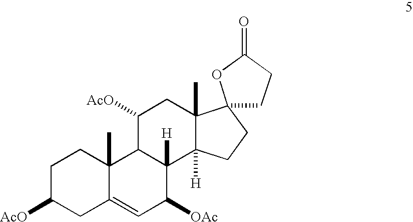
e) carbonylating a compound of Formula 5 to give a compound of Formula 6; 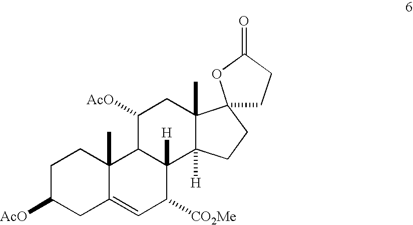
f) biotransforming a compound of Formula 6 to a compound of Formula 7b; 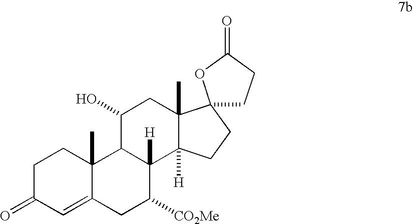
g) dehydrating a compound of Formula 7b to a compound of Formula 8;
h) oxidizing a compound of Formula 8 to eplerenone, Formula 9.

6. A process according to claim 1 for preparing steroid intermediates further comprising the steps:
a) reacting acetylene with a compound of Formula 1 to give a compound of Formula 2;
b) acetylating a compound of Formula 2 to give a compound of Formula 3;
c) hydroformylating a compound of Formula 3 to give a compound of Formula 4;
d) oxidizing a compound of Formula 4 to give a compound of Formula 5;
e) carbonylating a compound of Formula 5 to give a compound of Formula 6; and
f) biotransforming a compound of Formula 6 to a compound of Formula 7b.

7. A process according to claim 1 for preparing intermediates for the synthesis eplerenone comprising biotransforming a compound of Formula 11 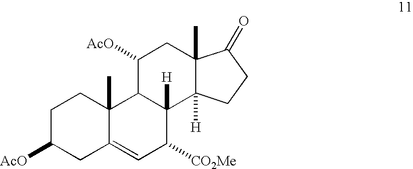
to a compound of Formula 12b.

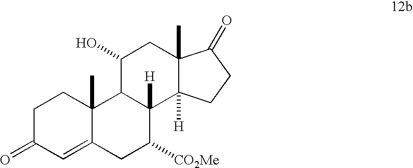

8. A process according to claim 7 for preparing eplerenone further comprising the steps:
a) reacting a compound of Formula 12b with acetylene to give a compound of Formula 13; 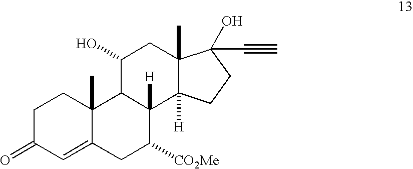
c) ketalizing a compound of Formula 13 to give a compound of Formula 14; 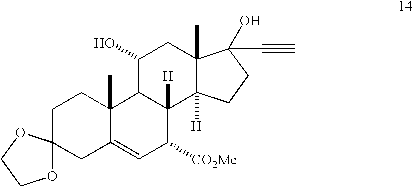
d) hydroformylating a compound of Formula 14 to give a compound of Formula 15; 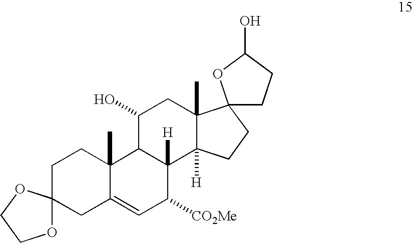
e) oxidizing a compound of Formula 15 to give a compound of Formula 16; 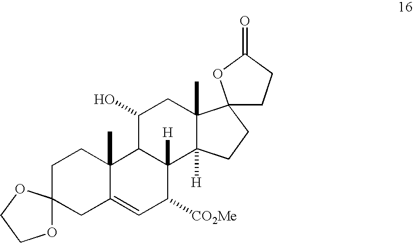
f) hydrolyzing a ketal of Formula 16 to give a compound of Formula 7b; 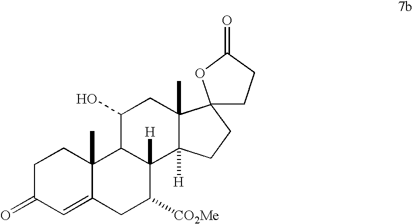
g) dehydrating a compound of Formula 7b to give a compound of Formula 8; 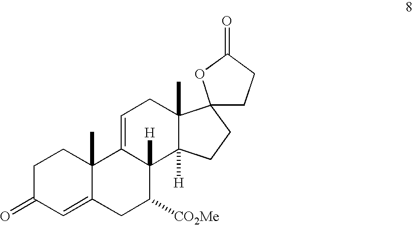
h) oxidizing a compound of Formula 8 to give elperenone, 9.

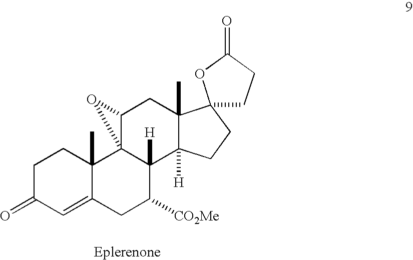

9. A process according to claim 1 for preparing steroid intermediates comprising biotransformation of a compound of Formula 27 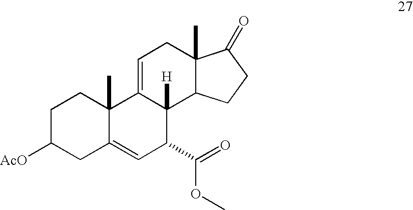
to a compound of Formula 28b.

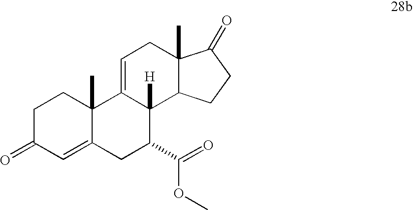

10. A process for preparing eplerenone according to claim 9 further comprising the steps:
a) acetylating a compound of Formula 25 to give a diacetoxy steroid compound Formula 26;
b) carbonylating a compound of Formula 26 to give a compound of Formula 27;
c) biotransforming a compound of Formula 27 to a compound of Formula 28b;
d) reacting a compound of Formula 28b with acetylene to give a compound of Formula 29; 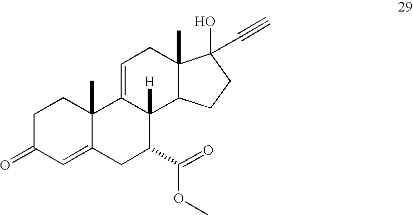
e) ketalizing a compound of Formula 29 to give a compound of Formula 30; 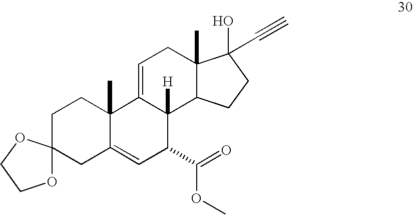
f) hydroformylating a compound of Formula 30 to give a compound of Formula 31; 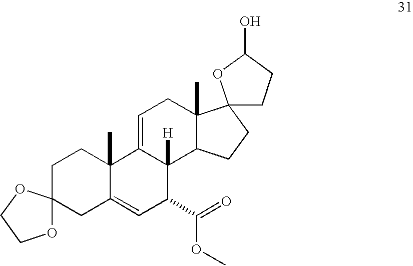
g) oxidizing a compound of Formula 31 to give a compound of Formula 32 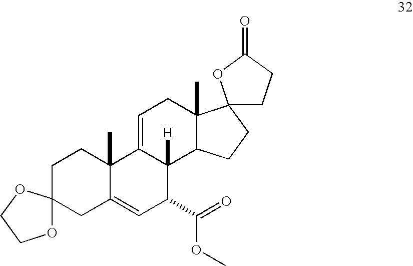
h) hydrolyzing a compound of Formula 32 to give a compound of Formula 8; 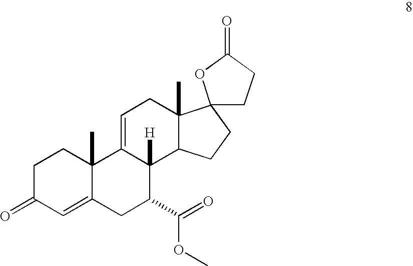
i) oxidizing a compound of Formula 8 to eplerenone, Formula 9.

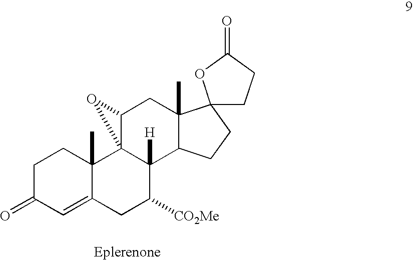

11. A process of preparing steroid intermediates according to claim 3 comprising biotransforming a compound of Formula 5 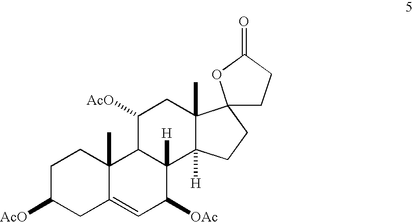
to compound of Formula 35a.

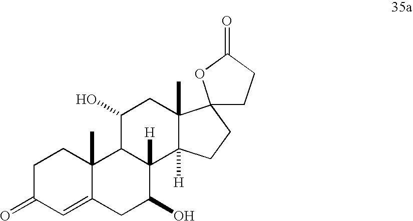

12. A process of preparing eplerenone according to claim 11 further comprising the steps:
a) dehydrating a compound of Formula 35a to a compound of Formula 36; 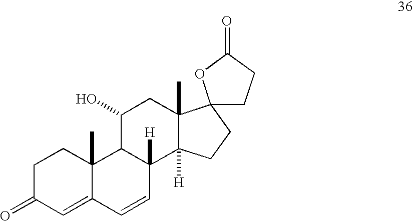
b) reacting a compound of Formula 36 with a cyanohydrin and subsequent hydrolysis to give a compound of Formula 7;
c) dehdrating a compound of Formula 7 to give a compound of Formula 8;
d) oxidizing a compound of Formula 8 to a compound of Formula 9, eplerenone.
